(19) United States
(12) Patent Application Publication
(10) Pub. No.: US 2016/0324487 A1
(43) Pub. Date: Nov. 10, 2016

(54) Wearable Personal Computer and Healthcare Devices

(71) Applicant: INTEL CORPORATION, Santa Clara, CA (US)

(72) Inventors: Mao GUO, Shanghai, 31 (CN); Junfeng ZHAO, Shanghai, 31 (CN); Michael P. SKINNER, San Jose, CA (US); Ke XIAO, Shanghai, 31 (CN); Jiamiao TANG, Shanghai, 31 (CN); Bin LIU, Shanghai, 31 (CN); Li DENG, Shanghai, 31 (CN)

(21) Appl. No.: 14/779,513

(22) PCT Filed: Nov. 27, 2014
(86) PCT No.: PCT/CN2014/092345

§ 371 Date: Sep. 23, 2015

(51) Int. Cl. A61B 5/00; G08B 5/22; A61B 7/00; A61B 5/107; A61B 5/11; H04Q 9/00; A61B 5/08

(52) U.S. Cl. CPC A61B 5/746; H04Q 9/00; G08B 5/224; A61B 5/6804; A61B 5/0022; A61B 5/0816; A61B 5/1072; A61B 5/486; A61B 5/1118; A61B 5/4815; A61B 5/7455; A61B 5/7405; A61B 7/00; H04Q 2209/43; A61B 2562/166; A61B 2562/0247; A61B 2562/0219; A61B 2562/04

(57) ABSTRACT

Embodiments described herein may fully integrate personal computing and health care into a wearable waistband having a length sensor, a pressure sensor, and a motion sensor; or into a wearable “mesh” having an array of sound sensors, which will create convenient and seamless access to a personal computer and biofeedback of the wearer. Such biofeedback from the waistband may include determining respiration rate, waist length, food quantity of a meal, sitting or sleep time, and frequency of visits to the bathroom. Such biofeedback from the mesh or array may include determining whether there is or has been damage or other issues of the heart, lungs, bones, joints, jaw, throat, arteries, digestive tract, and the like. Such biofeedback may also detect whether whether a person has an allergic reaction at a location, is drinking (and what volume of fluid), is walking, is jogging or is running.

### BACKGROUND

1. Field

Wearable circuit devices and the manufacture and structure of wearable health care devices, computers and circuit devices.

2. Description of Related Art

Existing personal computers cannot be comfortably worn on the human body, and nor do they provide biometric and tracking of physiological functions. Wearable devices such as watches, glasses can monitor vital signs (e.g., pulse) and/or work as personal assistant to access emails, Internet, and videos, but the computing capability or functionality of these devices is heavily constrained due to their small form factor. There are a number of non-trivial issues associated with fabricating such wearable devices.

### BRIEF DESCRIPTION OF THE DRAWINGS

The embodiments of the invention are illustrated by way of example and not by way of limitation in the figures of the accompanying drawings in which like references indicate similar elements. It should be noted that references to “an” or “one” embodiment of the invention in this disclosure are not necessarily to the same embodiment, and they mean at least one.

FIG. 1 is a schematic cross-section view of a portion of a wearable computer device capable of being worn around a person's waist.

FIG. 2 is a diagram illustrating a data transfer system for the device of FIG. 1 or a processor of the device of FIG. 1.

FIG. 3 is a schematic cross-section view of a buckle of the device of FIG. 1.

FIG. 4 is a schematic cross-section view of a portion of a sound sensor capable of being worn on and sensing sound from a person.

FIG. 5 is a schematic view of an array of multiple sound sensors of FIG. 4 worn by or mounted on a wearer.

### DETAILED DESCRIPTION

Embodiments described herein may fully integrate personal computing and health care into system or device such as (1) a belt or waistband (e.g., device 100 described below); or (2) a “mesh” or array of sound sensors (e.g., array 510 described below), which will create a convenient and seamless access to a personal computer and biofeedback. The wearable waistband may have a length sensor, a pressure sensor, and a motion sensor. Such biofeedback from the waistband may include determining respiration rate, waist length, food quantity of a meal, sitting or sleep time, and frequency of visits to the bathroom. The wearable “mesh” or array may have an array of sound sensors, and the biofeedback from the mesh or array may include determining whether there is or has been damage or other issues of the heart, lungs, bones, joints, jaw, throat, arteries, digestive tract, and the like.

Embodiments described herein have a form factor of waistband. They may be bigger than any other wearable device so that more functionality or capability can be integrated within them. Moreover, biosensors can be included in or on the waistband to monitor respiration rate, waist length, food quantity, sitting or sleep time, frequency of visits to the bathroom due to the devices working position on the body. This is compared to devices that are not worn on the waist and thus can not monitor these qualities or environments since they are farther away from the waist.

Some embodiments include a wearable computer system which has a “computer on a board,” such as on a printed circuit board (PCB) fully incorporated into a waistband to be worn by a person, “wearer” or “user”. A user of the device may be the wearer of the device or another person who puts the device on the wearer. Such a computer may be a developmental system for wearable devices, such as a system based on an Intel® Edison board (e.g., the Intel® Edison breakout board, chip or computer module. Some sensors (e.g., biosensors) are also built into the waistband to sense data that the computer can use to capture biofeedback such as respiration rate, waist length, food quantity, sitting/sleep time, and frequency to bathroom. Biofeedback outputs may be displayed on a mobile phone or other device after wireless transmission of the outputs to that device, such as by wireless local area network (WLAN), such as WiFi™. Biofeedback outputs may also be indicated by different vibration frequencies or vibration patterns over time, of a vibrator located on the device. Biofeedback outputs may also be indicated by different audio frequencies or audio patterns over time, of a speaker located on the device. Biofeedback outputs may be stored in a memory of the device (e.g., processor or part of the computer) and downloaded later to another device.

Some embodiments may include a universal serial bus (USB) interface to make data access to large amounts of data more efficient than existing wearable devices. The interface may be used to receive software or program or application instructions and/or updates. The interface may be used to transmit or download biofeedback data or alerts.

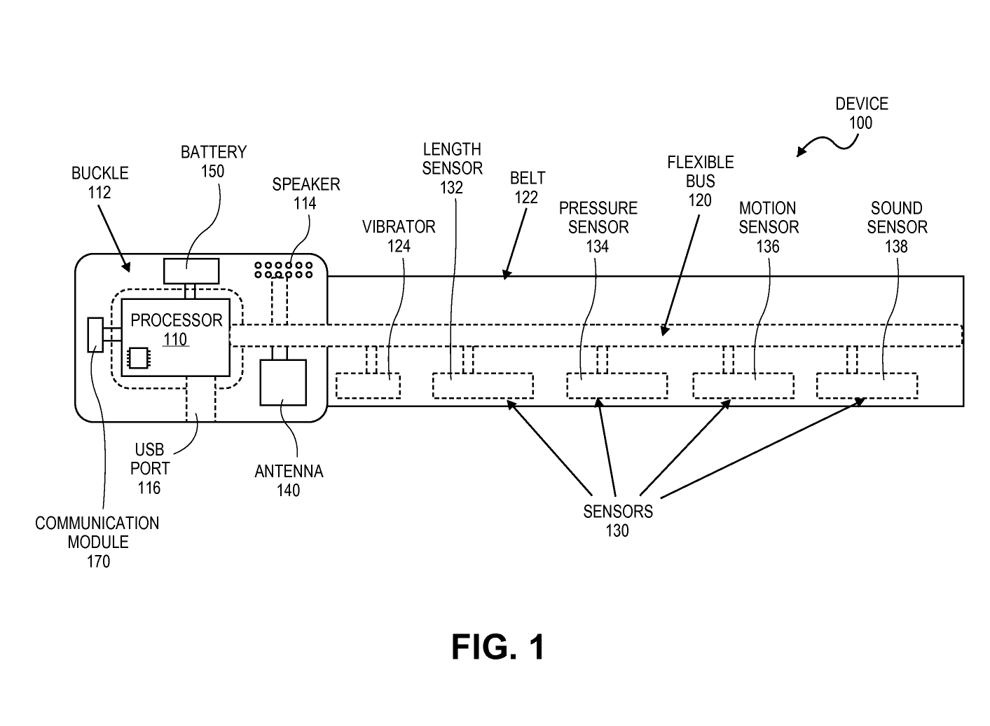

In some case, the belt may include a waistband (e.g., wide belt such as device 100), a processor (e.g., a computer on a board, such as an Intel® Edison board), biometric sensors, a vibrator, a speaker, a USB interface, and some flexible PCB (e.g., data bus) which transfers the data between the processor and the other parts (e.g., see FIG. 1).

The biometric sensors and USB interface may work as input/output (I/O) systems or devices of the wearable device (e.g., computer or processor). The vibrator and speaker may also work as I/O systems or devices of the wearable device.

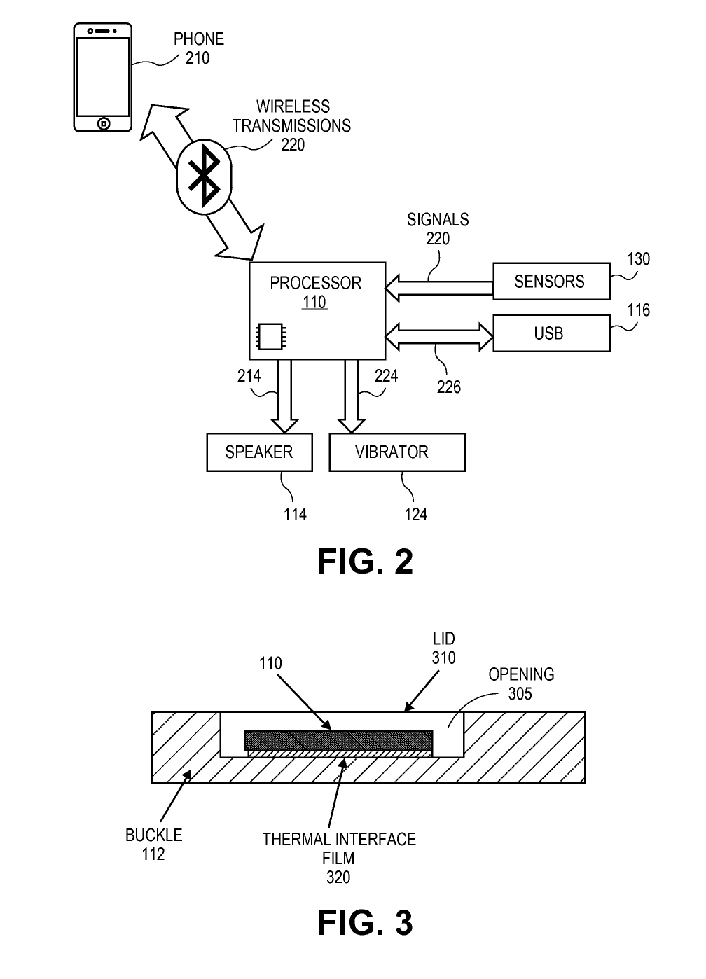

In some cases application software or programming installed on the processor (e.g., computer) and can access the I/O data and communicate with a smartphone, tablet or other device through wireless communication or technology (e.g., see FIGS. 1 and 2).

In some cases, conventional electronic device and clothing assembly technology and materials are used for device production. The processor can be fixed in a cavity of a metal buckle by some thermal interface film (e.g., see FIGS. 1 and 3). The biometric sensors can be easily installed in or on a leather belt because of their small size.

FIG. 1 is a schematic cross-section view of a portion of a wearable computer device 100 capable of being worn around a person's waist. Device 100 is shown having buckle 112 attached to belt 122. Buckle 112 includes or houses electronic components such as processor 110, universal serial bus 116 and speaker 114. In some cases, buckle 112 also includes or houses antennae 140, communication module 170 and/or battery 150. According to embodiments, antennae 140, communication module 170 and/or battery 150 may be part of processor 110 or may be separately housed in the buckle (from processor 110). Belt 122 includes bus 120, vibrator 124 and sensors 130. Sensors 130 are shown including light sensor 132, pressure sensor 134, motion sensor 136, and sound sensor 138. Sensors 130 may be described as biometric or physiological sensors.

In some cases, device 100 may not include or may exclude any one or more of universal serial bus 116, speaker 114, vibrator 124, light sensor 132, pressure sensor 134, motion sensor 136, and/or sound sensor 138 (e.g., they may be optional). In some cases, device 100 may not include or may exclude any one or more of antennae 140 and communication module 170 (e.g., they may be optional).

Device 100 may represent a piece of clothing, a waistband, a belt, a girdle, a support garment, or other device capable of housing or having the components of FIG. 1 attached to it. In some cases device 100 is a belt having a proximal end with buckle 112 and a distal end (not shown) for removably attaching to buckle 112, such as to “close” or “buckle” the device. For instance the distal end of belt 122 may be slidable into a portion of buckle 112, and buckle 112 may have a mechanism for locking or buckling the proximal end of belt 122 at a set length, to buckle 112. In some cases, the distal end of belt 122 may have openings or holes through which a post or portion of buckle 112 may extend to moveably lock the distal end of the belt at a location (e.g., latch) in the buckle.

When the distal end of belt 122 is locked or buckled to buckle 112, sensor 132 may sense the length of the perimeter or circumference that device 100 forms. When the distal end of belt 122 is locked to buckle 112, sensors 134 and 138 may have sensing mechanisms exposed and facing (e.g., orientated) inward, such as towards and exposed to a person wearing device 100.

Length sensor 132 can be configured to directly measure (e.g., sense) a length or changes in a length of the wearer of device 100. The length or changes can be used (e.g., by processor 110) to detect fast or large changes in a length of the wearers waist.

In some cases, processor 110 is configured or programmed (e.g., via the USB port or otherwise) to process or analyze output data sent to it by the length sensor. For example, waist length sensor application software or programming installed on the processor can access the I/O data from the length sensor (e.g., via bus 120). The processor can also store or record the waist length data in a timeframe, and give the wearer an alert (e.g., via speaker 114 or vibrator 124) if the waist length changes sharply, or transmit the data to another device using USB port 116 or wireless transmission (e.g., module 170).

The software or processor 110 can detect changes in the length sensor output data based on whether the length exceeds thresholds over time (or based on an algorithm) to detect or determine changes in the length of the wearer's waist. If the length is greater than a threshold, a too large alert can be transmitted to the speaker and/or vibrator on the device. The alert may also be transmitted to a mobile phone, tablet or other device through wireless communication or technology. Descriptions herein for wireless communication or technology may include short distance wireless (e.g., using short-wavelength ultra high frequency (UHF) radio waves in a frequency range of from 2.4 to 2.485 gigahertz (GHz) from fixed and mobile devices, such as Bluetooth®), or personal area network technology.

If the change in length (e.g., increase or decrease) over a period of time (e.g., a day, a week, two weeks, or a month) is greater than a threshold (e.g., 1 inch or 2 inches per month), a quick change alert can be transmitted to the speaker and/or vibrator on the device. The alert may also be transmitted to a mobile phone, tablet or other device through the wireless communication.

Length sensor 132 may be disposed within, attached to, or mounted within belt 122. Sensor 132 may be mounted by screws, adhesive, or otherwise. Sensor 132 may be electronically coupled to bus 120, such as to transmit electronic length output signals to processor 110. Sensor 132 may be a micro sensor or other length sensor that fits within belt 122. The location of sensor 132 may be a desired location for sensor 132 (e.g., on belt 122 or device 100 with respect to waist or organs of the wearer) to sense a length indicator (e.g., evidentiary length) of the body of the wearer, of an organ (e.g., waist) of the wearer.

Sensor 132 may be capable of outputting an electronic length signal on bus 120 and to processor 110, when a person is wearing device 100, such as during normal conditions in public, at home, in a bathroom, and/or in an office setting. Sensor 132 may sense an “environment” around or associated with device 100, such as a biometric (physiological) waist length environment of a person wearing the device. Sensor 132 may then output a signal on bus 120, to processor 110, based on the sensed environment. For example, sensor 132 may sense a total length of device 100 or belt 122 when worn by a person and the distal end of belt 122 is buckled or removably locked to buckle 112; and output data on bus 120 and to processor 110, representing that length. Sensor 132 may be able to measure a waist length or belt length of device 100, when it is around a person's waist or abdomen.

In some cases, sensor 132 extends along the entire length of belt 122, such as to detect where sensor (or belt 122) overlaps. In this way, sensor 132 (or processor 110) can detect the length of device 100 when device 100 is buckled. For example, sensor 132 may be capable of measuring a length of device 100 or non-overlapping distance of belt 122, when the distal end of belt 122 is buckled or removably locked at buckle 112. Processor may know or be programmed with a length of buckle 112 so that the length of device 100 can be determined, such as by adding the length of buckle 112 with the non-overlapping length of belt 122 (e.g., as detected by sensor 132).

Pressure sensor 134 can be configured to measure changes (e.g., variations) in abdomen pressure of the wearer of device 100. The changes can be used (e.g., by processor 110) to detect respiration of and a quantity of food ingested by the wearer of the device.

In some cases, processor 110 is configured or programmed (e.g., via the USB port or otherwise) to process or analyze output data sent by the pressure sensor. For example, pressure sensor application software or programming installed on the processor can access the I/O data from the pressure sensor (e.g., via bus 120). The processor can also store or record the pressure data in a timeframe, and give the wearer an alert (e.g., via speaker 114 or vibrator 124) if the pressure changes sharply, or transmit the data to another device using USB port 116 or wireless transmission (e.g., module 170).

The software or processor can detect changes in the pressure sensor output data based on whether the pressure exceeds a threshold (e.g., an increase or decrease of more than 1, 2 or 3 psi per 1-3 seconds) over time (or based on an algorithm) to detect or determine respiration rate. The rate can be communicated to the user or wearer (e.g., the person wearing the device) or another person, by being transmitted to a mobile phone, tablet or other device through the wireless communication.

The software or processor can detect changes in the pressure sensor output data based on whether the pressure exceeds thresholds over time (or based on an algorithm) to detect or determine an amount of drink and/or food eaten (e.g., consumed) by the wearer. If the amount consumed is greater than a threshold (e.g., a pressure increase by an average of 2, 5 or 10 psi per hour), a too much food alert can be transmitted to the speaker and/or vibrator on the device. The alert may also be transmitted to a mobile phone, tablet or other device through the wireless communication.

Pressure sensor 134 may be disposed within, attached to, or mounted within belt 122. Pressure sensor 134 may be mounted by screws, adhesive, or otherwise. Sensor 134 may be electronically coupled to bus 120, such as to transmit electronic pressure output signals to processor 110. Sensor 134 may be a micro sensor or other pressure sensor that fits within belt 122. Buckle 112 may include one or more inward openings “above” sensor 134, through which sensor 134 may sense pressure, such as an outward pressure of or that is caused by the body or organ (e.g., waist or belly) of a person wearing device 100 (e.g., when buckled or unbuckled). The location of sensor 134 may be a desired location for sensor 134 (e.g., on belt 122 or device 100 with respect to organs of the wearer) to sense an indicator (e.g., evidentiary pressure) of the body of the wearer, of an organ (e.g., waist) of the wearer, or of the stomach or belly of the wearer.

Sensor 134 may be positioned facing inward, such as towards a person wearing device 100. Sensor 134 may be capable of outputting an electronic pressure signal on bus 120 and to processor 110, when a person is wearing device 100, such as during normal conditions in public, at home, in a bathroom, and/or in an office setting. Sensor 134 may sense an “environment” around or associated with device 100, such as a biometric (physiological) waist pressure environment of a person wearing the device. Sensor 134 may then output a signal on bus 120, to processor 110, based on the sensed environment. For example, sensor 134 may sense a total pressure at one or more locations along the inner surface of device 100 or belt 122 when worn by a person and the distal end of belt 122 is buckled or removably locked to buckle 112; and output data on bus 120 and to processor 110, representing that or those pressure(s). It may also sense those pressure(s) and output that data when the distal end of belt 122 is not buckled or not removably locked to buckle 112. Sensor 134 may be able to measure a pressure incident upon sensor 134, from a person's waist or abdomen, when device 100 is worn by a person. In some cases, sensor 134 is one or more sensors disposed along the entire length of belt 122, such as to detect pressure where sensors 134 are located.

Motion sensor 136 (e.g. position sensor or accelerometer) can be configured to directly measure (e.g., detect) movement of the motion sensor 136, device 100 and/or a wearer of the device. The changes can be used (e.g., by processor 110) to detect or determine how long of a time (e.g., period) the wearers sits or sleeps.

In some cases, processor 110 is configured or programmed (e.g., via the USB port or otherwise) to process or analyze output data sent by the motion sensor. For example, motion sensor application software or programming installed on the processor can access the I/O data from the motion sensor (e.g., via bus 120). The processor can also store or record the motion data in a timeframe, and give the wearer an alert (e.g., via speaker 114 or vibrator 124) if the wearer should exercise or is not sleeping well, or transmit the data to another device using USB port 116 or wireless transmission (e.g., module 170).

The software or processor can detect whether the motion exceeds thresholds over a period of time (or based on an algorithm) to detect or determine no or minimal motion over the period of time. If the motion is below a threshold (e.g., less than 10 feet of movement, less than 50 steps, etc.) over a period of time (e.g., an hour, two hours or four hours), a not enough exercise alert can be transmitted to the speaker and/or vibrator on the device. The alert may also be transmitted to a mobile phone, tablet or other device through the wireless communication.

The software or processor can detect whether the motion exceeds thresholds over a period of time (or based on an algorithm) to detect or determine restless sleep over the period of time. If the motion is greater than a threshold (e.g., more than 50 feet of movement, more than 150 steps, etc.) over a period of time (e.g., 4 hours, 6 hours or 8 hours at night, such as between 10 pm and 6 am; or between midnight and 8 am), a not enough good sleep alert can be transmitted to the speaker and/or vibrator on the device. The alert may also be transmitted to a mobile phone, tablet or other device through the wireless communication.

Motion sensor 136 may be or include an accelerometer, a position sensor, a motion sensor, an orientation sensor, or any combination thereof. Motion sensor 136 may be disposed within, attached to, or mounted within belt 122. Sensor 136 may be mounted by screws, adhesive, or otherwise. Sensor 136 may be electronically coupled to bus 120, such as to transmit electronic motion output signals to processor 110. Sensor 136 may be a micro sensor or other motion sensor that fits within belt 122. Sensor 136 may sense motion or movement, such as a movement or change in location of device 100 that is motion or movement of the waist of a person wearing device 100 (e.g., when buckled). Sensor 136 may be capable of outputting an electronic motion signal on bus 120 and to processor 110, when a person is wearing device 100, such as during normal conditions in public, at home, in a bathroom, and/or in an office setting. The location of sensor 136 may be a desired location for sensor 136 (e.g., on belt 122 or device 100 with respect to organs of the wearer) to sense an indicator (e.g., evidentiary motion or movement) of the body of the wearer, of an organ (e.g., waist) of the wearer.

Sensor 136 may sense an “environment” around or associated with device 100, such as a biometric (physiological) location or movement environment of a person wearing the device, and then output a signal on bus 120, to processor 110, based on the sensed environment. For example, sensor 136 may sense or track 3 dimensional (3D) relative movement of a location of sensor 136 over time when device 100 is worn by a person; and output data on bus 120 and to processor 110, representing that or those 3D movement(s). Such motion may be periodically detected and reported to the processor such as once per second, 5 seconds, 30 seconds, minute, 5 minutes or 10 minutes. In some cases, sensor 136 is one or more sensors disposed along the entire length of belt 122, such as to detect motion where sensors 136 are located.

The output data from the length sensor 132 and pressure sensor 134 can also be used (e.g., by processor 110) to measure or sense when a wearer of the device goes to bathroom. The changes can be used (e.g., by processor 110) to detect or determine how often a wearer of the device goes to bathroom (e.g., over a period of time period). If the frequency (e.g., number of times over the period) is too small (e.g., as compared to a selected theshold), an alert can be sent to the wearer or user to remind the wearer to drink more water over time.

For instance, the length sensor can be used (e.g., by the processor) to detect or determine that the device has been unbuckled or removed (e.g., there is an error, no length, or an infinite length measurement) such as when the wearer goes to bathroom. Also, the pressure sensor can be used (e.g., by the processor) to detect or determine that the device has been unbuckled or removed (e.g., there is an error, or no pressure measurement) such as when the wearer goes to bathroom. These detections can be used independently or in combination to detect or determine how often the wearer goes to bathroom over a period of time.

In some cases, the processor is configured or programmed to process or analyze output data sent by the length and pressure sensors. For example, bathroom sensor application software or programming installed on the processor can access the I/O data from the length and pressure sensors (e.g., via bus 120). The processor can also store or record the bathroom data in a timeframe, and give the wearer an alert (e.g., via speaker 114 or vibrator 124) if the wearer should drink more water, or transmit the data to another device using a USB port or wireless transmission (e.g., module 170).

The software or processor can detect whether the number of times going to the bathroom exceeds thresholds over a period of time (or based on an algorithm) to detect or determine not enough frequency. If the frequency is below a threshold (e.g., less than 2 times, less than 4 times, etc.) over a period of time (e.g., 8 hours, 12 hours, a day), a need more water alert can be transmitted to the speaker and/or vibrator on the device. The alert may also be transmitted to a mobile phone, tablet or other device through the wireless communication.

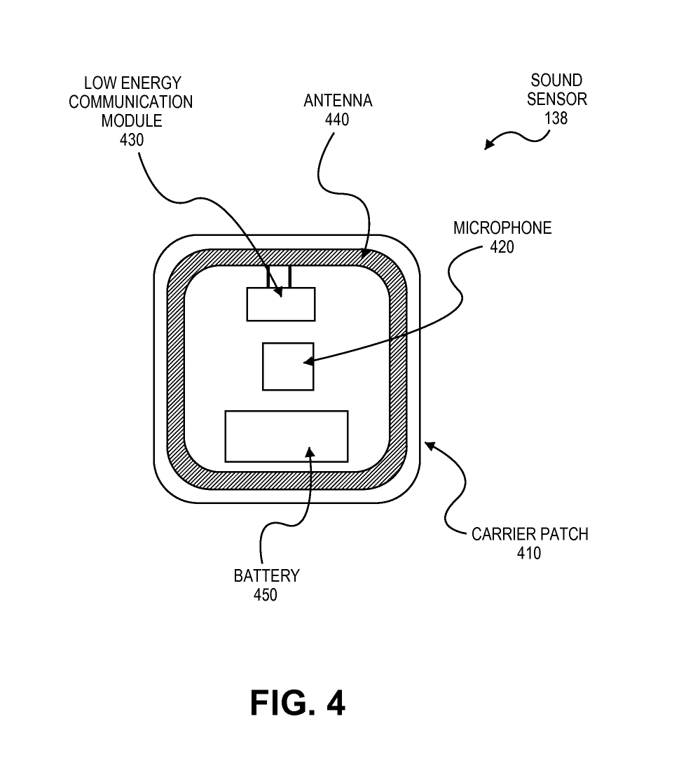

Sound sensor 138 may be or include a microphone, electro mechanical transducer, audio detector, or any combination thereof. Sensor 138 may be a sensor as described below for FIG. 4. Sound sensor 138 may be disposed within, attached to, or mounted within belt 122. Sound sensor 138 may be mounted by screws, adhesive, or otherwise. Sensor 138 may be electronically coupled to bus 120, such as to transmit electronic sound output signals to processor 110. Sensor 138 may be a micro sensor, micro microphone, small microphone or other sound sensor that fits within belt 122. Buckle 112 may include one or more inward openings “above” sensor 138, through which sensor 138 may sense outward sound, such as a sound of or that is caused by the body (e.g., at the waist or other location) of a person wearing device 100 (e.g., when buckled).

Sensor 138 may be positioned facing inward, such as towards a person wearing device 100. Sensor 138 may be capable of outputting an electronic sound signal on bus 120 and to processor 110, when a person is wearing device 100, such as during normal conditions in public, at home, in a bathroom, and/or in an office setting. Sensor 138 may sense an “environment” around or associated with device 100, such as a biometric (physiological) sound environment of a person wearing the device. Sensor 138 may then output a signal on bus 120, to processor 110, based on the sensed environment. For example, sensor 138 may sense an incident sound or audio vibration at one or more locations along the inner surface of device 100 or belt 122 when worn by a person and the distal end of belt 122 is buckled or removably locked to buckle 112; and output data on bus 120 and to processor 110, representing that or those sound(s). Sensor 138 may be able to measure a sound incident upon sensor 138, from, a person's waist or abdomen when device 100 is worn by a person.

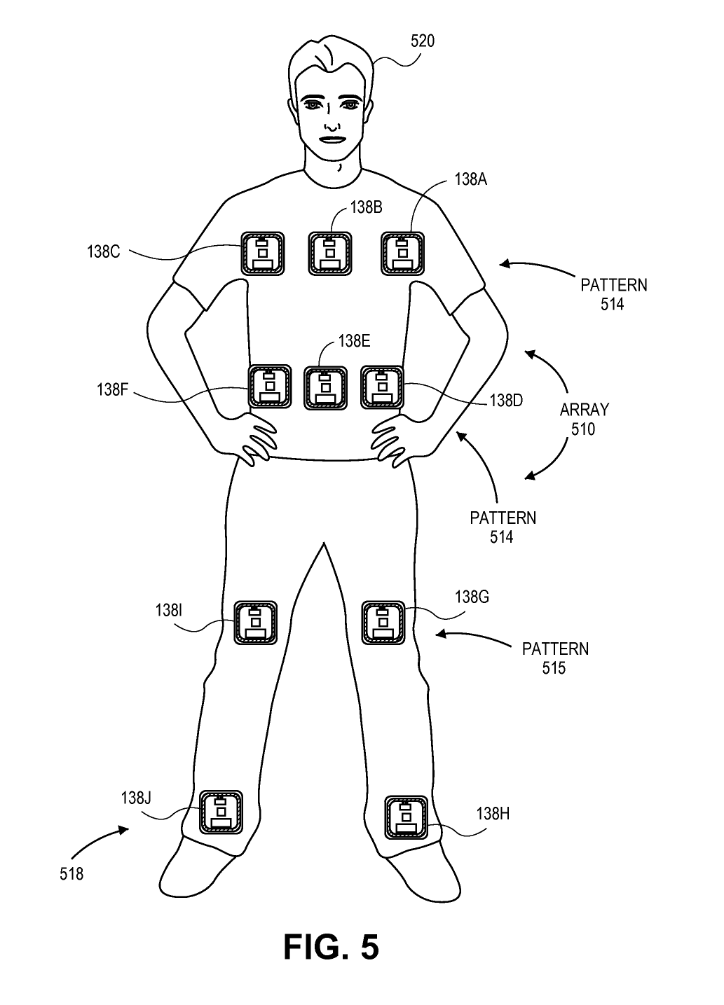

In some cases, sensor 138 is one or more sensors disposed along the entire length of belt 122, such as to detect sound where sensors 138 are located. In some cases, sensor may represent a number of sensors disposed along the a piece of clothing, on a person's skin, in an array of sensors, or on a device other than device 100, such as to detect sound where sensors 138 are located. Such as described below with respect to FIGS. 4-5.

In some cases, the location(s) of sensor 138 may be a desired location for sensor 138 (e.g., on belt 122 or device 100 with respect to organs of the wearer) to sense an indicator sound (e.g., evidentiary sound) at an organ of the body of the wearer (e.g., by processor 110). Based on the indicator sound, the processor may determine whether there is or has been damage or other issues noted herein related to the organ. If there has been damage or other issues, the processor can send an alert signal, such as to an alarm or other device. In some cases, the processor can give the wearer an alert (e.g., via speaker 114 or vibrator 124, or otherwise), or transmit the alert signal (and output data) to another device using a USB port or wireless transmission (e.g., module 170 or 430). The desired location, indicator sound and alert may be those described below with respect to FIGS. 4-5.

Speaker 114 may be attached, fixed, or permanently mounted within or on buckle 112. Speaker 114 may be mounted by screws, adhesive, or otherwise. Speaker 114 may be electronically coupled to bus 120, such as to receive electronic audio (e.g., alert) output signals from processor 110. Speaker 114 may be a micro speaker, small speaker, audio device, or other sound transducer for making sound that fits within buckle 112. Buckle 112 may include openings over speaker 114, through which sound created by speaker 114 may pass away from the buckle or wearer.

Speaker 114 may be positioned facing outward, such as away from a person wearing device 100. Speaker 114 may be positioned facing upwards, such as towards the head of a person wearing device 100. Speaker 114 may be an “alarm” device capable of outputting an alert audio signal that can be heard by a person wearing device 100, such as during normal conditions in public, at home, in a bathroom, and/or in an office setting.

Vibrator 124 is shown disposed within, attached to, or mounted within belt 122. Vibrator 124 may be mounted by screws, adhesive, or otherwise. Vibrator 124 may be electronically coupled to bus 120, such as to receive electronic vibration (e.g., alert) output signals from processor 110. Vibrator 124 may be a micro vibrator, vibrating device, or other vibration transducer for making vibrations that fits within belt 122.

Vibrator 124 may be an “alarm” device capable of outputting an alert vibrations or vibration sensations that can be felt by a person wearing device 100, such as during normal conditions in public, at home, in a bathroom, and/or in an office setting.

USB port 116 may be disposed in or attached to buckle 112. USB port 116 may be a female universal serial bus interface or port to interface with a USB capable device (e.g., having a male USB port) as known in the industry.

Port 116 may be coupled or electrically attached to processor 110. Port 116 may be capable of receiving and transmitting data and power signals between processor 110 and another USB device such as a flash drive, mobile phone, pad computer, other computer or other device, as known in the industry or a USB port.

Port 116 may be attached, fixed, or permanently mounted within or on buckle 112. Port 116 may be mounted by screws, adhesive, or otherwise. Port 116 may be electronically coupled to processor 110 and or to a bus that interfaces with processor 110.

Some embodiments port 116 may be a USB interface to provide processor 110 with data access to large amounts of data, software, program or application instructions for programming processor 110 to perform the input, processing, creating, generating and other processes described herein that processor 110 and/or device 100 perform. It may also be an interface to transmit or download biofeedback data or alerts.

Bus 120 may be a flexible data bus capable of bending with belt 122 without being damaged. Bus 120 may also be capable of being part of belt 122 that is buckled or removably locked to buckle 112 without being damaged. Bus 120 may be a flexible printed circuit board having conductive traces for transmitting the electronic signals. In some cases, bus 120 may be a flexible data bus or computer bus mounted within buckle 112 and belt 122.

In some embodiments, the bus includes a flexible PCB of a polyimide (PI) film or polyethylene terephthalate (PET) film as a dielectric and supporting material; and copper traces on the film. An adhesive may be used to reliably attach the copper traces to PI or PET film.

Bus 120 can be any subsystem adapted to transfer data within the device 100. Bus 120 can be a plurality of computer buses and include additional circuitry to transfer data and generally facilitate inter-component communication. Bus 120 may be capable of communicating electronic signals (e.g., as noted herein) between processor 110 and other components of device 100, including speaker 114, vibrator 124, sensors 130, port 116, antennae 140 and module 170. Bus 120 may be capable of transmitting electronic audio signals from processor 110 to “drive” speaker 114. Bus 120 may be capable of transmitting electronic vibration signals from processor 110 to vibrator 124, capable of driving the vibrator. Bus 120 may be capable of transmitting output signals from sensors 130 to processor 110. Bus 120 may be capable of transmitting output and receiving input signals between port 116 and processor 110. Bus 120 may be capable of transmitting output signals to module 170 from processor 110. Bus 120 may be capable of receiving output signals from antennae 140 at processor 110.

In some cases, bus 120 may be a computer bus capable of communicating electronic signals between processor 110 and the other components of device 100, using various known communication protocols or existing data transfer interfaces. Such protocols or interfaces include a peripheral component interconnect (PCI), PCIExpress, industry standard architecture (ISA), accelerated graphics port (AGP), universal serial bus (USB) or other bus capable of interfacing with and communicating data to and from processor 110. In some cases bus 120 may use a wireless technology to communicate the signals described herein (e.g., see modules 170 and 430).

In some cases, processor 110 may be computer on a board, or another processor or computer capable of fitting within buckle 112 and processing, interpreting, generating controlling electronic signals between processor 110 and the other components of device 100, such as using bus 120 or as otherwise described herein. Processor 110 may be a small computer that has a programmable memory and a central processing unit (CPU). It can be preprogrammed handwave logic and/or software before being mounted or located in the buckle, or can be programmed through antennae 140 or port 116. It may be programmed with a software application for performing the the input, processing, creating, generating and other processes described herein that processor 110 and/or device 100 perform.

Such a computer on a board may be or include an an Intel® Edison board or other tiny computer offered as a development system for wearable devices. In some cases, processor 110 may be the same size and shape as a secure digital (SD) card and contain a dual-core CPU executing at 500 megahertz (MHz) communicating via wireless technology and WLAN. In some cases, processor 110 may be or include a 22 nanometer (nm) dual core CPU. In some cases, processor 110 may be or include bigger and thicker than a standard SD card.

In some cases, processor 110 may be or include a microprocessor development board such as a printed circuit board containing a microprocessor and the minimal support logic needed for an engineer to become acquainted with the microprocessor on the board and to learn to program it. It also served users of the microprocessor as a method to prototype applications in products.

Unlike a general-purpose system such as a home computer, processor 110 may include little or no hardware dedicated to a user interface. It may have some provision to accept and run a user-supplied program, such as downloading a program through USB port 116 to a memory (e.g., to flash memory). It may accept and run a user-supplied program, such as downloading a program through a serial or USB port to flash memory, or some form of programmable memory in a socket. In some cases, processor 110 may include a read only memory (ROM) based built-in machine language monitor, “debugger” or a “Keyboard Input Monitor”.

Processor 110 may be attached, fixed, or permanently mounted within or on buckle 112, such as is known for mounting or “packaging” a processor. Processor 110 may be mounted as shown in FIG. 3. Processor 110 may be electronically coupled to bus 120, such as to receive and transmit electronic signals as noted herein. Processor 110 may fit within buckle 112. Buckle 112 may include openings above processor 110, through which air may pass to cool processor 110.

Processor 110 may be capable of communicating electronic signals (e.g., as noted herein) through bus 120 to other components of device 100, including speaker 114, vibrator 124, sensors 130, port 116, antennae 140 and module 170. Processor 110 may be capable of creating (e.g., generating) and transmitting electronic audio signals from to “drive” speaker 114, and vibrator 124. Processor 110 may be capable of receiving and processing (e.g., interpreting) output signals from sensors 130. Processor 110 may be capable of creating and transmitting output signal to (and receiving and processing input signals from) between port 116. Processor 110 may be capable of creating and transmitting output signals to module 170. Processor 110 may be capable of receiving and processing output signals from antennae 140.

Processor 110 may include any suitably programmed processor, computer processor, signal processor, microprocessor, “chip”, or other central processing unit (CPU) as know in the industry. In some embodiments, the processor 110 may be a primary processor such as a microprocessor or central processing unit (not shown). Processor 110 may be or include electronic components such as a processor, a data storage containing an operating system and application software for execution by the processor. Processor 110 may include a circuit board upon which a programmable processor; random access memory (RAM); read only memory (ROM) or other memory that interfaces with the processor to execute instructions are mounted and interfaced. Processor 110 may be implemented in firmware, software or hardware (e.g., as an application-specific integrated circuit). In normal conditions, processor 110 may be configured (e.g., with hardware or logic) and/or programmed (e.g., with software or instructions) to perform the input, processing, creating, generating and other processes described herein that processor 110 and/or device 100 perform.

Processor 110 may be coupled to and include a memory, and the processor may be configured to execute instructions (e.g., computer program instructions) stored in the memory. The memory may store a software application or instructions for performing the processes described herein such as the input, processing, creating, generating and other processes described herein that processor 110 and/or device 100 perform. The memory may be described as a machine (e.g., computer) readable storage medium. The memory may be a non-volatile memory from which the instructions are loaded to a volatile memory (e.g., RAM) during execution by the processor. Moreover, the processor and memory may include or be coupled to bus 120, battery 150 and other components noted in FIGS. 1-2.

FIG. 1 shows battery 150 mounted independently of processor 110 in buckle 112, and electrically coupled to power processor 110. According to other embodiments, battery 150 may be part of processor 110 such as by being mounted on the same board or PCB as processor 110 (e.g., may not be separately housed in the buckle from processor 110). In some cases, the battery may be part of the processor chip, die or package.

Battery 150 may be a lead acid, lithium, rechargeable, removable, “watch” or other battery capable of powering processor 110. In other cases, battery 150 is electrically a capacitor or super capacitor which functions as a battery but effectively has no internal resistance so it charges and discharges rapidly. It may also be capable of powering other components of device 100. Battery 150 may be coupled to processor 110 to provide electrical power sufficient to power processor 110 for more than 8 or 12 hours. In some cases, battery 150 is only coupled to processor 110, and processor 110 provides necessary and sufficient power to speaker 114, vibrator 124, sensors 130, bus 120, antennae 140, communication module 170, and USB 116. In other cases, battery 150 is electrically coupled to processor 110, and one or more of speaker 114, vibrator 124, sensors 130, bus 120, antennae 140, communication module 170, and USB 116.

Battery 150 may be attached, fixed, or permanently mounted within or on buckle 112. Battery 150 may be mounted by screws, adhesive, or otherwise. Battery 150 may be electronically coupled to processor 110 and or bus 120, such as to provide power (and ground) signal to them. Buckle 112 may include one or more removable covers for accessing battery 150.

FIG. 1 shows communication module 170 mounted in or on buckle 112, and electrically coupled to receive signals from processor 110 for transmission. According to other embodiments, communication module 170 may be part of processor 110 such as by being mounted on the same board or PCB as processor 110 (e.g., may not be separately housed in the buckle from processor 110). In some cases, module 170 may be part of the processor chip, die or package. Module 170 may be electronically coupled to bus 120 (or other electrical hardware connections), such as to receive electronic communication output signals from processor 110.

Communication module 170 may be a wireless communication antennae, transmitter or transceiver capable of encoding, modulating and transmitting wireless signals, such as radio signals, WLAN, wireless technology, and the like. Communication module 170 may implement any of a number of wireless standards or protocols, including but not limited to Wi-Fi™ (Institute of Electrical and Electronics Engineers—IEEE 802.11 family), Worldwide Interoperability for Microwave Access (WiMAX) (IEEE 802.16 family), IEEE 802.20, long term evolution (LTE), Enhanced Voice-Data Optimized or Enhanced Voice-Data Only (Ev-DO), Evolved High-Speed Packet Access (HSPA+), Bluetooth, the like, and derivatives thereof, as well as any other wireless protocols that are designated as third generation (3G), fourth generation (4G), fifth generation (5G), and beyond.

FIG. 1 shows antennae 140 mounted in or on buckle 112, and electrically coupled to send received signals to processor 110. According to other embodiments, antennae 140 may be part of processor 110 such as by being mounted on the same board or PCB as processor 110 (e.g., may not be separately housed in the buckle from processor 110). In some cases, antennae 140 may be part of the processor chip, die or package. Antennae 140 may be electronically coupled to bus 120 (or other electrical hardware connections), such as to transmit electronic communication output signals from processor 110.

Antennae 140 may be a wireless communication antennae or receiver capable of receiving, demodulating and decoding wireless signals, such as radio signals, WLAN, wireless technology, and the like. Antennae 140 may implement any of a number of wireless standards or protocols, including those mentioned above for module 170.

FIG. 2 is diagram illustrating a data transfer system for device 100 or processor 110. FIG. 2 shows processor 110 sending and receiving wireless transmissions 220 from phone 210. In some cases, phone 210 may be a smartphone, mobile phone or cell phone; a laptop, notebook, tablet, or desktop computer; a personal digital assistant (PDA); a monitor; a set-top box or entertainment control unit. In further implementations, phone 210 may be any other electronic device that processes data.

Transmissions 220 may be radio transmissions, WLAN transmissions, cell transmissions, wireless technology transmissions, or other transmission systems known in the industry, including those mentioned above for module 170. Transmissions 220 may be transmitted by module 170 and received by antennae 140. Transmissions 220 may be transmissions transmitted by module 170 and received by antennae 140.

Processor 110 is shown receiving signals 220 from sensors 130. Signals 220 may be signals from sensors 130 (e.g., sensors 132, 134, 136 and 138) as described above for bus 120. Processor 110 is shown receiving signals 226 from USB port 116. Signals 226 may be signals to and from port 116 as described above for port 116. Signals 226 may be signals as known in the industry for communicating between a processor and a USB port. Processor 110 is shown transmitting or sending signals 220 to vibrator 124. Signals 124 may be signals described above for sending to vibrator 124 on bus 120. Processor 110 is shown sending signals 214 to speaker 114. Signals 214 may be signals described above for sending to speaker 114 upon bus 120.

FIG. 3 is a schematic cross-section view of buckle 112. FIG. 3 shows buckle 112 having opening 305 in which processor 110 is disposed or attached to buckle 112. FIG. 3 shows lid 310 over or housing processor 110 within opening 305. Lid 310 may be permanently or removably attached to sides of opening 305 or buckle 112.

FIG. 3 shows thermal interface film 320 attaching CPU 310 to the bottom surface of opening 305, or to buckle 112. Processor 110 may be low power processor or computing system that does not generate much heat. Film 320 may be an adhesive film designed to maintain adhesion of chip 110 to buckle 112 when the film is heated by the processor. The film may be designed to survive heating by the processor.

In some cases film 110 conducts heat between a processor and the buckle sufficiently to use the buckle as a thermal sink or cooling device for the processor. In some cases, film 320 may be a film capable of thermally isolating processor 110 from buckle 112 so as not to cause the heat generated by processor 110 from making the buckle to be too warm or hot for a person wearing device 100. Film 320 may also isolate processor 110 so that the processor does not overheat. It can be appreciated that other adhesives or mechanisms can be used to mount the processor in the opening.

Some embodiments include a process for determining a biometric reading using device 100. Such a process may include locating device 100 around a waist of a person. Locating the waistband may include locating sensors 130 (e.g., sensors 132, 134, and 136 (and optionally 138)) at desired locations around the waist. Next, the sensors may sense a biometric environment and output electronic output signals based on the sensed environment. The output signals may be communicated by bus 120 from the sensors to processor 110 where they are received. Next, the processor may output an alert signal to an alarm (e.g., speaker 114, vibrator 124, and/or a wireless signal such as to cellular phone 210) if an electronic output signal is greater than a threshold or includes a profile. If an electronic output signal is greater than a threshold or includes a profile, the processor may determine or detect a respiration rate, waist length, food quantity of a meal, sitting or sleep time, or frequency of visits to the bathroom based on the output signals. In response to receiving the alert signal, the alarm (e.g., speaker 114, vibrator 124, and/or cellular phone 210) may alerting the person wearing device 100 of the alert signal.

FIG. 4 is a schematic cross-section view of a portion of a sound sensor capable of being worn on and sensing sound from a person. FIG. 4 shows sound sensor 138 such as a sensor unit or “patch” having a sound sensor such as a microphone. Sound sensor 138 may be integrated into device 100 (e.g., as noted above), can be independently worn by a wearer, or can be part of a “mesh” or array 510 of sound sensors (e.g., see FIG. 5). Sensor 138 is shown having carrier patch 410 upon which houses electrical components such as microphone 420, low energy communication module 430, antennae 440, and battery 450. In some cases, the sound sensor includes the carrier patch, the microphone 420, and a wired or wireless a communication module. Sensor 138 and patch 410 are discussed further below. FIG. 4 is further described below.

FIG. 5 is a schematic view of multiple sound sensor units worn by or mounted on a wearer. FIG. 5 shows sound sensors 138A-138J in a mesh or array 510 worn by a person or wearer 520. Array 510 includes abdomen or chest pattern 512, waist pattern 514, left leg pattern 515 and right leg pattern 516. Pattern 512 includes sensors 138A, 138B and 138C. Pattern 514 includes sensors 138D, 138E and 138F. Pattern 515 includes sensors 138G and 138H. Pattern 516 includes sensors 138I and 138J. Array 510 may be described as a “mesh” or “array” of microphones or a microphone mesh system. Array 510 is shown including 10 microphone units, patches, or sound sensors. It can be appreciated that array 510 conclude more or fewer sound sensors, such as by including 3, 6, 7, or more than 10 microphones. Array 510 may include 15, 20 or 30 sound sensors.

In some examples, sensors of array 510 also include sensors similar to 138A to 138J, but on the back, or posterior side of the wearer 520. In this case, any reference to one or more sensors includes reference to the corresponding sensor directly behind it and on the back side of the wearer.

In some cases, array 510 may include pattern 512, 514, 515, and/or 516. In some cases, only pattern 512 or pattern 514 are present. In some cases only patterns 515 and 516 are present. In some cases, only patterns 512 and 514 are present. Sensor 138 or array 510 may include or use one or more sound sensors 138 to sense the sounds at one or more locations so that those sounds can be captured and analyzed (e.g., by a processor) to monitor biometric activity, such as to monitor or detect heartbeat, digestion, respiration, body movement, damage to the body, or other issues. FIG. 5 is further described below.

Currently, human body sound listening is mainly limited to reactive modes and limited sound sources. Doctors mainly listen to the person's lung during medical check or health check to see whether the person has a breath problem. This kind of limited body sound listening typically only happens in short period and not in-situ. Human body sound listening is also typically limited to certain body position for certain single purposes, such as during a visit to a doctor office. For example, the lungs are often listened to by a doctor while a knee joint is seldom listened by a doctor. Embodiments herein may include or utilize a wearable microphone mesh (an array or matrix of multiple microphones (e.g., sensors 138) scattered around the body) to monitor human body's (e.g., of a wearer of device 100, sensor 138 or array 510) health, safety and activities, by listening to the sounds received by sound sensors from the human body such as heartbeat, blood flow, breath and joint movement. The human body may generate various sounds (either audible or non-audile or non-audio) and these sounds can be “listened to” (e.g., detected) with sensitive microphones (e.g., sensors 138). There may be some correlation between the sounds and human body's health and activity, By listening and analyzing these sounds (e.g., filtering to capture a frequency of interests such as biometric frequency or biometric “indicator sound”), certain aspects, biometric activity or issues of the human body's health and biometric activities can be determined (e.g., by a processor).

Some embodiments include or utilize multiple microphones (e.g., multiple sensor 138 such as in array 510) to sense the sounds at multiple locations (e.g., selected locations) so that all those sounds can be captured and analyzed (e.g., by a processor) so that the system is multifunctional. Such embodiments may be described as a sound listening based multiple-function health, biometric and activity monitor system (e.g., array 510) such as to monitor or detect heartbeat, digestion, respiration and body movement, such as to detect a biometric indicator or activity sound of damage to or other issues of the organs.

Some embodiments include or utilize a full body wearable device or mesh having an array of sound sensors (e.g., multiple sensor 138 such as in array 510). The multiple microphones system (e.g., a processor) can analyze sounds by comparing the sound difference listened to or detected by microphones at different locations (e.g., selected locations). This system (e.g., FIG. 5) can be used to monitor (e.g., using a processor) something that can not be monitored by a single microphone (e.g., FIG. 1 or 4). This system (e.g., based on these sounds) can also be used as feedback to noise cancelation so as to focus into a specific sound.

Sensor 138 or array 510 may include or use one or more sound sensors 138 to sense the sounds at one or more locations so that those sounds can be captured and analyzed (e.g., by a processor) to monitor biometric activity, such as to monitor or detect heartbeat, digestion, respiration, body movement, damage to the body, or other issues. Based on the detections, a processor to receive signals from Sensor 138 or array 510 may send an alert signal, such a to an alarm or other device. In some cases, the processor can give the wearer an alert (e.g., via speaker 114 or vibrator 124, or otherwise), or transmit the alert signal (and output data) to another device using a USB port or wireless transmission module 170 or 430).

In more detail, FIG. 4 shows sensor 138 in the form of patch 410 which houses electrical components such as microphone 420, low energy communication module 430, antennae 440, and battery 450. FIG. 4 shows communication module 430 and antennae 440 mounted in or on patch 410, and electrically coupled to receive signals from microphone 420 for transmission by antennae 440. According to other embodiments, communication module 430 and antennae 440 may be mounted on the same board or PCB as microphone 420. Communication module 430 may be a wireless communication antennae or transmitter similar to module 170 (e.g., to send wireless output signals of sound sensed by sensors 138). Module 430 may be attached, fixed, or permanently mounted within or on patch 410 similar to how module 170 is mounted in buckle 112. Module 170 may be electronically coupled to components of patch 410.

In some case, low energy communication module 430 and antennae 440 communicate audio output data or output data representing sound heard by microphone 420 to a processor, similar to signals 220, sensor output data on bus 120 sent to processor 110 by low energy wireless communication. In some case, battery 450 provides power to microphone 420 or components of sensor 138 similar to battery 150 providing power to processor 110.

In some cases, patch 410 may include microphone 420, battery 450 or a capacitor to power sensor 138 (e.g., see battery 150), a tow energy communication module 430 such as wireless technology and antenna 440. The antenna can also be used to charge the battery or capacitor as an option. All these elements are attached to one side of a patch, which can be a plastic or fabric piece. The electrical interconnection between the electronic components or silicon chip such as the microphone can be using conductive adhesive, a PCB, a flexible bus (such as bus 120), traces, and/or solder material.

There may be adhesive on the other side of the patch or on the same side of the patch as the openings to the microphone, depending on how the patch will be attached, either on the clothes (other side, e.g., of array 510 or the mesh) or on the human skin directly (same side). The microphone may listen to the sounds and send the output signals for the sounds either in a raw form (e.g., without signal processing) or after being processed (such as by audio circuitry or a processor) to a master device such as a processor or a smart phone via the low energy communication, The sounds may be further processed by the processor or the smart phone. The processing may include signal filtering based on sound frequency that will filter out noise and retain the sounds (e.g., frequencies of sound) of interests. For example, the blood flow induced sounds can be fitted by retaining frequency around 1 hertz (Hz) to detect an indicator sound indicating an amount of blood flow. Heart rate can be measured through analyzing or filtering to retain sounds around 1 hertz (Hz) to detect an indicator sound for heart rate.

FIG. 4 shows a wireless form of sensor 138, but sensor 138 can be wired too for connection to processor 110. If it is wired, the low energy communication module 430 and/or antenna 440 and/or the battery 450 can be excluded or saved. In such case, output signals from and power to the sensors can be provided (e.g., conducted) using coated conductive wires that can be part of fabric of the clothes or the stitch line where the cloth is stitched together to make the “mesh” or array. The electrical interconnection between the coated wire and the sensor 138 can be a conductive adhesive. In the manufacturing process of the array or mesh, some conductive adhesive with solvent that can react with the coating on the wire can remove the coating on the wire and form electrical connection of the wire to the component of the sensor.

In some cases, multiple sensors 138 will make up a “mesh” or array 510. FIG. 5 shows an example of the microphone mesh system that includes 10 microphone units shown in FIG. 4, In FIG. 5 the sensor locations demonstrate the possible location on a human body. In some cases, the sensors are not necessary on the surface of the clothes; they can be attached onto the skin or the inner side of the clothes, so that the mesh or array 510 is invisible to others. Depending on electrical connection solutions, the sensors of the array or mesh can be either wired or wireless. If wired, the electrical connection can be done with the process described above. If wireless, there is no physical electrical connection (e.g., wires) between the sensors required.

In accordance with some embodiments, each sensor 138 can work independently (as a single sensor or as part of array 510). For example, the sensor located (e.g., a selected location) closest to the heart (e.g., sensor 138B) can monitor the heartbeat while the sensor located (e.g., a selected location) close to the lungs (e.g., sensor 138E on the wearer's back) can monitor the respiration. These sensors can also work together. When they work together, the array 510 or system can recognize the relative location of the sensor automatically (e.g., selected locations). This can be important for wearer or user experience, because that person can simply attach the sensor on the location he likes (e.g., a selected location as noted) and the system such as a processor or smartphone identifies where the sensor is located on the wearer (e.g., selected locations). This can be done with one or multiple processes. The first is to use wireless positioning for the sensor. Here, each sensor can transmit or receive wireless signals. The signal strength is related to the distance between two sensors. The relative location (e.g., at selected locations) can be calculated based on the calculation of the signal strength measured between any two of these sensors. The second method is to use sound strength to identify the relative location of the sensor. In this method, the sensor will generate a sound with certain strength, while other sensor will receive a sound with various strengths depending on the distance between two sensors. Based on sent compared to received sound strength between any two of all the sensors, the distance can be calculated and identify the relative location of all the sensors. If these sensors are wired together instead of wireless connected, the relative location can be identified by measuring the wiring resistance between two sensors. The longer the wire, the higher the resistance. This can assist in locating the sensors (e.g., at selected locations).

When the mesh or array 510 of sensors work together (e.g., more than one of their output signals are considered or compared by a processor), some functions can be realized that can't be realized with a single sensor. For example, in FIG. 5, if the left arm of the human body is hit by something or attacked by somebody, sound from the sensors (e.g., all array 510 or pattern 512) can be compared to determine that the location of the left arm has been hit. All the sensors will receive sounds with different strength, so that the rough position where the body is hit can be determined. For example, sound from sensor 138A can be compared to sound from sensor 138C, J, H or other sensors to determine that the location of the left arm has been hit (e.g., based on a found impact sound at sensor 138A, but not at sensor 138C, J, H). It is valuable when a human body encounters an accident. The information can help doctors to judge the injury location and severity.

The sensors 138 may be connected to each other or to a processor or controller for analyzing the data, such as to detect biometric activity. For example, in some cases a controller or processor (e.g., similar to processor 110) may be located in one of the sensors, in a separate device, or in device 100 (e.g., such as by being processor 110). Each sensor 138 may communicate with that processor, through wired or wireless communication. In some cases, some of the sensors communicate through wire communication, while some communicate wirelessly.

Wire communication may include communicating through wires, a date bus (e.g., similar to bus 120) or through a multi-wire or coax type cable with the controller. Wires may exist as part of the material of the mesh or clothing. The wires may be separate than the material and adhered to strung along the material of the mesh or clothing. In some cases the wires may be woven through the mesh or material of the clothing. The wires may communicate data from the sensors to the processor. Such data may include sound data and output signals described for sensor 138. The wired communications signals may include signals described for signals 220.

Wiring in the mesh may form an antennae to communicate signals from the wired sensors to another processor, such as processor 110. Wires in the mesh may provide superior transmission of data from the sensors than antennae 440, and/or better reception of data for the sound sensors than that of module 430. This may be due to the larger, less interference, or heavier material that can be used for wires in the mesh. It is also considered that wires within or woven through the mesh may be used to receive or generate power or electricity, such as for powering the sound sensors.

In some cases, patch 410 may be a housing or device upon which are mounted (or within which are mounted) microphone 420, low energy communication module 430, antennae 440, and battery 450. Patch may be made of PCB, substrate, cloth, cotton, polyester, rayon, denim, plastic, polymer, rubber, metal, alloy, or other material capable of housing or having the components of FIG. 4 attached to it. The patches may have various shapes and sizes. For example, the patch may be or have a perimeter up to or less than two square inches, 1 square inch, ½ square inch, an ¼ of a square inch, a tenth of a square inch. The patch may have the shape of a square, a rectangle, a triangle, a circle, an oval, and the like. The patch may have a thickness of a quarter of an inch, 3/16 of an inch, an ⅛ of an inch, 1/16 of an inch, a 1/20 of an inch.

In some cases, microphone 420 may be a microphone, electro mechanical transducer, audio detector, or any combination thereof. Microphone 420 may be configured to directly measure (e.g., detect) sound or frequencies from 0.1 Hz-20 KHz from the skin (e.g., internal sounds) of a wearer of the device. In some cases the frequencies are from 0.1 Hz-5 KHz. The changes can be used (e.g., by a processor) to monitor (e.g., detect or determine) biometric activity, such as to monitor or detect heartbeat, digestion, respiration and body movement.

Sensor 138 may be a micro sensor, micro microphone, small microphone or other sound sensor that fits as noted, such as for an array or “mesh”, and may have an opening within which to mount such a microphone. Patch 410 may include one or more openings above or below sensor 138, through which sensor 138 (or microphone 420) may sense sound, such as a sound of or that is caused by the body (e.g., at the waist or other location) of a person wearing the sensor, such as for array 510.

Sensor 138 may be positioned or facing (e.g., orientated) towards or to sense sound inward, such as towards a person wearing the sensor, or as otherwise noted, such as for array 510. Sensor 138 may be capable of outputting an electronic sound signal to a processor such as mounted in one of sensor 138, separately, in a separate “patch”, or as noted for array 510, when a person is wearing the sensor, such as during normal conditions in public, at home, in a bathroom, and/or in an office setting.

Sensor 138 may sense an “environment” around or associated with a device (such as for device 100, array 510, or as otherwise noted), such as a biometric (physiological) sound environment of a person wearing the device. Sensor 138 may then output a signal to a processor as noted, such as for array 510, based on the sensed environment. For example, sensor 138 may sense an incident sound or audio vibration at one or more locations along the inner surface of patch 410 when worn by a person, or as otherwise noted, such as for array 510. Sensor may then output data to a processor, or as otherwise noted, such as for array 510, representing those sounds.

In some cases, processor 110 (or another processor, such as mounted in one of sensor 138, separately, or in a separate “patch”) is configured or programmed (e.g., via a USB port or otherwise) to process or analyze output data sent by the sound sensor (e.g., as wired or wireless output signals of sensors 138 as noted), such as to detect biometric activity of the wearer. For example, sound sensor application software or programming installed on the processor can access the I/O data from the sound sensor (e.g., via wired or wireless communication, or as otherwise noted, such as for array 510). The processor can also store or record the sound data in a timeframe, and give t le wearer an alert (e.g., via speaker 114 or vibrator 124, or otherwise) such as noted below for FIGS. 4 and/or 5, or transmit the data to another device using a USB port or wireless transmission module 170 or 430).

One of the patches may include a processor and thus be the “host” patch while the other processors are considered “slaves”. In some cases, more than one processor exists to process the signals. In some cases, more than one host exists and has a subset of the other patches as its “slave”. In some cases, a processor that is part of the mesh or clothing processes the signals from the sensors of the mesh and sends data to processor 110 for analysis. Processor 110 may thus provides alerts or outputs described with respect to sensor 138, but based on the sensors of pattern 510.

One or more sensor(s) 138 may be attached or mounted directly on a persons skin, such as at desired locations noted herein. In some cases, sensors 138 are attached to an inner surface of a piece of clothing, such as at desired locations noted herein. In some cases, sensors 138 are integrated into or in between layers of piece of clothing, such as at desired locations noted herein. The clothing can be any type of shirt, jacket or T-shirt (e.g., pattern 512; or 512 plus 514, either with optional wrist sensors); pants or shorts (e.g., pattern 515; or 515 plus 514); underwear (e.g., pattern 512; 512 plus 514; all patterns; 515; or 515 plus 514); girdle, waistband or belt (e.g., pattern 514; or 514 plus sensor 138I and G); turtleneck sweater, scarf or chocker (e.g., pattern 512; sensor 138B; sensors 138A and C; or sensor 138A or C), and the like. The cloth of the clothing can be any type of material including cotton, polyester, rayon, denim, plastic, polymer, rubber. In some cases, the mesh may include or be array 510 of sound sensors attached to the torso and legs of a wearer, either directly onto the skin or onto (1) a shirt and pants, or (2) a single piece of clothing to be worn by the wearer. In some cases, the mesh may include or be array 510 of sound sensors attached a single piece of clothing to be worn against (e.g., tightly against the skin) of the torso and legs. Such a single piece of clothing may be a leotard, “unitard”, overalls, body suit, “onezie”, jumper, or the like.

It is also considered that other ones of sound sensors 138 (or patterns thereof) may be included on a hat, beanie, mask, earmuffs, scarf, or other clothing on or around the head, such as at desired locations noted herein. The sensors may also make detections similar those described herein for pattern 512, sensors located on a collar of a shirt of turtleneck sweater, or sensors to sense the head. For instance, in some cases, one or more sensors may be mounted in the neck of a shirt or a “turtleneck” sweater to listen to the carotid artery, breathing, spine, etc.

The desired location(s) may be selected by the wearer or user of device 100 or array 510 based on or so that the sound sensor(s) are located to directly sense or indirectly sense (e.g., through triangulation) sound from an organ of the wearer of the device or array. The location of the sensor (e.g., on the skin, in clothing, or otherwise) can be selected to locate the sensor at, over, adjacent to, or for triangulation at a specific location where sound is expected to be heard, such as from an organ. Such sound may be include a biometric (physiological) environment or biometric activity of a person wearing the device, and and the sensor may output an output signal based on the sound or the sensed environment. In some cases, such sound may be described as or include an indicator (e.g., evidentiary) sound at the desired location (e.g., selected location), and the output signal may include the indicator sound (e.g., from the organ or a part of the organ). The indicator sound may be considered a biometric (physiological) environment or biometric activity of a person wearing the device.

In some cases, a single sensor may be located at a desired location (e.g., selected location) to sense an indicator (e.g., evidentiary) sound at or from an organ (or a part of the organ). This may include locating the sensor over or above the organ (e.g., at the desired location) to “directly” sense the indicator sound from the organ. In some cases, 2, 3, 4 or more sensor will be located at desired locations (e.g., selected locations) to sense an indicator (e.g., evidentiary) sound at or from an organ (or a part of the organ) by triangulation. This may include locating 2 or 3 sensors around the organ (or a part of the organ), but not over or above the organ, and triangulating the sound heard by those sensors to “indirectly” sense indicator sound at the organ. This may be done by locating one sensor over the organ; locating 2 or 3 sensors around the organ; and triangulating the sound heard by those sensors for indicator sound at the organ. In some cases, 2, 3, 4 or more of the sensors will be used to perform noise cancellation, such as to cancel sounds other than the indicator sound or sounds that are not from the organ. This may be done by locating the sensors as noted for triangulating.

An “organ” may describe an entire organ, a part an organ, or a specific location of an organ. The organs may include the heart, lungs, bone joints, jaw, mouth, nose, throat, arteries, digestive tract, liver, kidneys, bladder, intestines, stomach, pancreas, other organs, and the like. Thus, sensors at one or more desired locations may sense or detect indicator sound (from the organ) and based on that indicator sound determine whether there is or has been damage to or other issues related to the organ. For example, based on output signals from sounds detected by sensors at one or more organs, a processor may detect from the output signals whether the sounds detected include an indicator sound.

An indicator sound may be detected by determining (e.g., by a processor) that the sound detect from the output signals exceeds a threshold or includes a “profile” sound. An indicator sound (e.g., biometric indicator sound or a biometric activity) may be detected by determining that the sound detect is or includes: (1) sound in a frequency range that has an amplitude or volume greater than a threshold; (2) sound from (1) that is greater than the threshold within a selected period of time; (3) sound from (1) that has an average that is greater than the threshold within a selected period of time; or (4) sound that has a “profile” such as by having a selected frequency or time based profile over a selected period of time (e.g., such as a “popping”, “crunching”, “heart beat”, or “breathing” sound). Based on this, the processor may determine whether there is or has been damage or other issues noted herein related to the organ. If there has been damage or other issues, the processor can send an alert signal, such a to an alarm or other device. In some cases, the processor can give the wearer an alert (e.g., via speaker 114 or vibrator 124, or otherwise), or transmit the alert signal (and output data) to another device using a USB port or wireless transmission (e.g., module 170 or 430).

For example, one or more sensors may be used to listen for an indicator sound (e.g., a profile sound) at specific location of a body joint, such as to detect popping sound or other indicator/profile sound for a damaged ligament, muscle, cartilage, meniscus, and/or a lack of an amount of synovial fluid. In some cases a popping sound in the joint may indicate damaged meniscus or cartilage, such as of the knee, shoulders, elbow, ankle, etc. In some cases, such a sound may indicate such damage to the spine or bulging discs in the spine. In some case, such a sound may indicate a hip injury, joint problem, improper operation, or ligament flexor.

In some cases, the location may be with regards to the heart. For example sensor 138B may be located (e.g., on the skin, in clothing, or otherwise) at a desired location (e.g., selected location) directly above or over the heart (organ) to directly sense an indicator sound of the heart (or a part of the heart). Also, sensors of pattern 512; or pattern 512 plus 514 may be located (e.g., on the skin, in clothing, or otherwise) at a desired locations (e.g., selected locations) around the heart (e.g., proximate to the heart but not directly over the heart) to indirectly sense an indicator sound of the heart (or a part of the heart) such as by triangulation. In some cases, the desired location or organ may be the carotid artery, left heart valve, right heart valve, left heart ventricle, right heart ventricle, aorta, artery or vein of the heart.

In some cases, the location may be with regards to a large artery or vein away from the heart, such as an artery in the torso, leg, arm, neck, or abdomen. For example, sensors may be located (e.g., on the skin, in clothing, or otherwise) at a desired location directly above or over a large artery or vein away from the heart, such as an artery or vein (organ) in the torso (sensor 138A, B, C, D, F), leg (sensor 138I or G), arm (sensor 138C or A), neck (any of sensor 138A-D), or abdomen (any of sensor 138A-F) to directly sense an indicator sound of those organs (or a part of those organ). Also, sensors may be located at a desired locations around the heart (e.g., proximate to the heart but not necessarily directly over the heart) to indirectly sense an indicator sound of large artery or vein away from the heart, such as an artery or vein in the torso (sensors 138A, B, C, D and F), leg (sensor 138I and J; or G and H), arm (sensor 138C, F and B; or A, D and B), neck (sensor 138A-D), or abdomen (sensors 138A-F) to directly sense an indicator sound of those organs (or a part of those organ) such as by triangulation.

For the heart or a large artery or vein away from the heart, the indicator sound may be a sound amplitude, profile, frequency, change in frequency, heart beat frequency, heart beat pattern (e.g., electrocardiogram—EKG), heart beat profile, or range of frequency that indicates an amount of blood flow, speed of blood flow, blockage of blood flow, thrombosis, plaque, pulse, or change for any of a ventricle, valve, aorta, artery or vein of the heart or vasculature adjacent to the heart, that exceeds a threshold or includes a profile, such as noted herein.

Such indicator sounds may be used to detect damage to the heart or other issues such as, stretching, tear, softening or blockage in the ventricle, valve, aorta of the heart. Such indicator sounds may be used by the processor to detect damage to the vasculature or other issues such as a thrombosis, plaque or blockage in an artery or vein of the heart; or adjacent to the heart, such as in a leg, arm, neck, etc. Based on this, the processor may determine whether there is or has been damage or other issues noted above; and if so, send a heart or vasculature alert.

For example, the blood flow induced sounds can be fitted by retaining frequency around 1 Hz (e.g., 0.9 Hz to 1.1 Hz; or 0.8 Hz to 1.2 Hz) to detect an indicator sound indicating an amount of blood flow. If there is not enough sound amplitude or volume in at this frequency, an alert signal may be sent to indicate low/high blood flow, low/high blood pressure, heart damage or heart issues. Heart rate can be measured through analyzing or filtering to retain sounds around 1 Hz (e.g., 0.8 Hz to 1.2 Hz; or 0.7 Hz to 2 Hz) to detect an indicator sound for heart rate. If there are not enough sound amplitude or volume peaks in at this frequency, an alert signal may be sent to indicate low/high heart beat, low/high blood flow, heart damage or heart issues.

In some cases, such indicator sounds may be greater than a threshold volume (e.g., 3-5 Decibel (dB), or 5-10 dB from the heart) and have a threshold frequency between 0.5 Hz and 180 Hz. The threshold frequency for normal heart rate, elevated heart rate and excessive heart rate may be determined in this frequency range and may depend on the age of the wearer. Based on the frequency the processor may detect normal heart rate, elevated heart rate or excessive heart rate and send an alert signal indicating the detected heart rate.

In some cases, the location may be with regards to the lungs. For example, sensor 138B; 138D and E; pattern 512; pattern 514; or pattern 512 plus 514 may be located (e.g., on the skin, in clothing, or otherwise) at desired locations directly above or over the lungs; or one or more nodes of the lungs to directly sense an indicator sound of those organs (or a part of those organs). Also, those same sensors or pattern(s) may be located (e.g., on the skin, in clothing, or otherwise) at a desired locations around the lungs to indirectly sense an indicator sound of those organs (or a part of those organs) such as by triangulation. The desired locations may include the left upper lung nodes, left central lung nodes, left lower lung nodes, right upper lung nodes, right central lung nodes, right lower lung nodes, and the like. Similarly, the sensors can be used to directly or indirectly sense an indicator sound of other breathing organs such as the larynx, the trachea or throat.

For the lungs, larynx, trachea or throat, the indicator sound may be a sound amplitude, profile, frequency, change in frequency, beat frequency, or range of frequency that indicates an amount of air flow, speed of air flow, blockage of air flow or change for any of a lung node, larynx, trachea or throat, that exceeds a threshold or includes a profile, such as noted herein. Such indicator sounds may be used by the processor to detect damage to the lungs or other issues, such as abnormal slowing or speeding up of air-flow capability. Such indicator sounds may be used to detect damage to the lungs, other breathing organs. Based on this, the processor may determine whether there is or has been damage or other issues noted above; and if so, send a lung or breathing alert.

In some cases, such indicator sounds may be greater than a threshold volume (e.g., 3-5 dB, or 5-10 dB from the lungs) and have a threshold frequency between 0.2 Hz and 3 Hz. The threshold frequency for normal breathing rate, elevated breathing rate and excessive breathing rate may be determined in this frequency range and may depend on the age of the wearer. Based on the frequency the processor may detect normal breathing rate, elevated breathing rate or excessive breathing rate and send an alert signal indicating the detected breathing rate.

In some cases, the location may be with regards to bone joints.

For example, sensor 138I may be located at desired locations directly above or over the the right knee to directly sense an indicator sound of that organ (or a part of that organ). Also, sensors 138I and J; or 138I, J, F and E may be located at desired locations around that organ to indirectly sense an indicator sound of that knee (or a part of that organ) such as by triangulation.

In another example, sensor 138G may be located at desired locations directly above or over the the left knee to directly sense an indicator sound of that organ (or a part of that organ). Also, sensors 138G and H; or 138G, H, D and E may be located at desired locations around that organ to indirectly sense an indicator sound of that knee (or a part of that organ) such as by triangulation.

For the knees, the indicator sound may be a sound amplitude, profile, frequency, change in frequency, grinding sound or popping sound that indicates a resistance of movement, blockage of movement, tear in tissue, or sprain in tissue for a joint, ligament, meniscus, or muscle of that knee (e.g., medial collateral ligament, patella bone, lateral collateral ligament, patella ligament, anterior cruciate ligament, posterior cruciate ligament), that exceeds a threshold or includes a profile, such as noted herein. Such indicator sounds may be used by the processor to detect damage to a joint, ligament, meniscus, or muscle of that knee. Based on this, the processor may determine whether there is or has been damage or other issues noted above; and if so, send a knee joint alert.

In some cases, such indicator sounds may be greater than a threshold volume (e.g., 5-10 dB, or 10-20 dB from the knee) and have a profile sound such as a “popping” or “crunching” sound profile. In some cases, such indicator sounds may also have a threshold frequency between 0.5 Hz and 3 Hz; or of a walking, jogging or running footstep frequency. Based on the sound profile the processor may detect ligament, cartilage, bone, tendon, sprain, swelling or other knee damage, and send an alert signal indicating the detected damage.

For example, sensor 138C may be located (e.g., on the skin, in clothing, or otherwise) at desired locations directly above or over the the right shoulder to directly sense an indicator sound of the right shoulder (or a part of that organ). Also, sensors 138C, D and F may be located (e.g., on the skin, in clothing, or otherwise) at a desired locations around that shoulder to indirectly sense an indicator sound of that shoulder (or a part of that organ) such as by triangulation.

In another example, sensor 138A may be located at desired locations directly above or over the the left shoulder to directly sense an indicator sound of that shoulder (or a part of that organ). Also, sensors 138A, D and B may be located at a desired locations around that shoulder to indirectly sense an indicator sound of that shoulder (or a part of that organ) such as by triangulation.

Sensor 138F may be located at desired locations directly above or over the the right hip to directly sense an indicator sound of that organ (or a part of that organ). Also, sensors 138F, E and I may be located at desired locations around that organ to indirectly sense an indicator sound of that hip (or a part of that organ) such as by triangulation.

In another example, sensor 138D may be located at desired locations directly above or over the the left hip to directly sense an indicator sound of that organ (or a part of that organ). Also, sensors 138D, E and G may be located at desired locations around that organ to indirectly sense an indicator sound of that hip (or a part of that organ) such as by triangulation.

A sensor may be located at the desired location on or over the right wrist to directly sense an indicator sound of the right wrist (or a part of that organ). Also, sensors 138C; 138F; and 138E and a sensor over the right wrist may located be desired locations around that organ to indirectly sense an indicator sound of that right wrist, (or a part of that organ) such as by triangulation.

In another example, a sensor may be located at the desired location on or over the left wrist to directly sense an indicator sound of that organ (or a part of that organ). Also, sensors 138A; 138D; and 138E and a sensor over the left wrist may located be desired locations around that organ to indirectly sense an indicator sound of that wrist (or a part of that organ) such as by triangulation.

For the shoulder, hip or wrist, the indicator sound may be a sound amplitude, profile, frequency, change in frequency, grinding sound or popping sound that indicates a resistance of movement, blockage of movement, tear in tissue, or sprain in tissue for a joint, ligament, meniscus, or muscle of that shoulder, hip or wrist, that exceeds a threshold or includes a profile, such as noted herein. Such indicator sounds may be used by the processor to detect damage to a joint, ligament, meniscus, or muscle of that shoulder, hip or wrist. Based on this, the processor may determine whether there is or has been damage or other issues noted above; and if so, send a shoulder, hip or wrist joint alert.

In some cases, such indicator sounds for the shoulder, hip or wrist may be greater than a threshold volume (e.g., 5-10 dB, or 10-20 dB from the shoulder, hip or wrist) and have a profile sound such as a “popping” or “crunching” sound profile. Based on the sound profile the processor may detect ligament, cartilage, bone, tendon, sprain, swelling or other shoulder, hip or wrist damage, and send an alert signal indicating the detected damage.

In some cases, the location may be with regards to the jaw, tongue, teeth, nose, mouth, and larynx. For example, sensor 138B, A, C, or a sensor over the neck, such as in collar, may be located (e.g., on the skin, in clothing, or otherwise) at desired locations to directly sense an indicator sound of the jaw, tongue, teeth, nose, mouth, or larynx. Also, pattern 512; 138C and B; 138B and A; 138A and C; 138C, a sensor on the neck, and 138B; 138B, sensor on the neck, and 138A; 138C, sensor on the neck, 138A, may be located (e.g., on the skin, in clothing, or otherwise) to indirectly sense an indicator sound of the jaw, tongue, teeth, nose, mouth, or larynx.

The sensors around the neck, such as in a collar, may be used to detect sounds expected from the location of the jaw, tongue, and teeth. Such sounds may include popping sounds, chewing sounds, low frequency repetitive sounds to indicate chewing, swallowing or eating issue. Such sounds may be used to detect whether a persons jaw or chewing is abnormal, such as by detecting “popping” in the jaw, this might indicate temporomandibular joint disorders (TMJ) or other abnormal jaw function.

In some cases, such indicator sounds for the jaw, tongue, and teeth may be greater than a threshold volume (e.g., 5-10 dB, or 10-20 dB) and have a profile sound such as a “popping” or “crunching” sound profile. In some cases, such indicator sounds may also have a threshold frequency between 0.5 Hz and 3 Hz; or of a chewing, biting, teeth grinding, or swallowing frequency. Based on the sound profile the processor may detect ligament, cartilage, bone, tooth, tendon, sprain, swelling or other jaw, tongue, and teeth damage, and send an alert signal indicating the detected damage.

In some cases, one or more of the desired locations above may be selected to listen for an indicator sound from the mouth, jaw, tongue, and teeth to detect a chewing, swallowing or eating issue or issue with one of those organs.

For these organs, the indicator sound may be a sound amplitude, profile, frequency, change in frequency, grinding sound or popping sound that indicates a resistance of movement, blockage of movement, tear in tissue, or sprain in tissue for a joint, ligament, meniscus, or muscle of the jaw, tongue, or teeth, that exceeds a threshold or includes a profile, such as noted herein. Such indicator sounds may be used by the processor to detect damage to a joint, ligament, meniscus, or muscle of that jaw, tongue, or teeth. Based on this, the processor may determine whether there is or has been damage or other issues noted above; and if so, send jaw, tongue, or teeth alert.

In other cases, for these organs, the indicator sound may be a sound amplitude, profile, frequency, change in frequency, beat frequency, or range of frequency that indicates an amount of air flow, speed of air flow, blockage of air flow or change for the nose, mouth, or larynx, that exceeds a threshold or includes a profile sound, such as noted herein. Such indicator sounds may be used by the processor to detect damage to the nose, mouth, or larynx, such as, abnormal slowing or speeding up of air-flow capability. Such indicator sounds may be used to detect damage to the nose, mouth, or larynx, other breathing organs. Based on this, the processor may determine whether there is or has been damage or other issues noted above; and if so, send a nose, mouth, larynx or breathing alert.

In some embodiments, indicator sounds with respect to different organs may be used to detect a type of activity of a person, such as whether they are walking, jogging, running, and the like. For instance, joint indicator sounds, footfall indicator sounds, and breathing indicator sounds of the joints, ankles and lungs (optionally also the heart; or nose and mouth) may be used by the processor to detect whether a wearer of array 510 is walking, jogging or running The processor may determine the frequency or speed of indicator sounds; combined with the average volume of the footfalls over a period such as 10 seconds, 30 seconds, 1 minute or 5 minutes to identify whether the wearer of the array is walking, jogging or running In some cases, such indicator sounds may be greater than threshold volumes and have a threshold frequency between 0.5 Hz and 3 Hz. The threshold volume for walking (e.g., 30-40 decibels—dB) may be lower (e.g., by between 2 and 5 times) than for jogging (e.g., 40-60 dB) which is lower (e.g., by between 2 and 5 times) than for running (e.g., 60-100 dB). In some cases, joint indicator sounds, footfall indicator sounds, and breathing indicator sounds are all compared to the frequency and volume thresholds above. In some cases, when a person runs, the sounds of the joints and breath could be different than those of another activity or state of the wearer. Detecting whether a wearer of array 510 is walking, jogging or running may include the processor also detecting the heart beat rate of the user is at a rate typical for walking, jogging or running; and combining that detection with the other walking, jogging and running indicator sounds or detections mentioned above.

Detecting whether a wearer of array 510 is walking, jogging or running may increase the judgment capability of the array to determine other indicator sounds or detections. In some examples, whether the wearer is walking, jogging or running may be combined by the processor with heart indicators sounds to detect a dangerous level of exertion of the heart, dangerous heartbeat rate, a heart attack or other heart issues. In some examples, whether the wearer is walking, jogging or running may be combined with combined by the processor with joint indicator sounds to detect damaged joints or tissue.

In some embodiments, drinking indicator sounds with respect to the mouth, throat, and/or esophagus that exceed a threshold or include a profile may be used to detect whether a person is drinking fluid and a volume of fluid drank over a period of time. For instance, throat swallowing indicator sounds (and optionally also mouth sucking indicator sounds or esophagus fluid indicator sounds) may be used by the processor to detect that a wearer of array 510 is drinking fluid and is drinking a volume of 0.25, 0.5, 0.75, 1 or 2 cups per 2 or 5seconds. The processor may determine the frequency or speed of these indicator sounds; combined with the average volume of the indicator sounds over a period such as 2, 3, 5 or 10 seconds to identify whether the volume of fluid being drank over that period. In some cases, the processor may determine that such indicator sounds may be greater than a threshold volume, have a certain length (e.g., profile sound such as a sound of a person swallowing) and have a threshold frequency between 0.5 Hz and 3 Hz. The threshold volume may be greater than (e.g., 20-40 dB) and higher frequencies may indicate greater volume being drank. Based on the drinking indicator sounds, the processor may determine how much fluid the wearer drinks over a longer period such as an hour, 4 hours, or a day. In some examples, whether the wearer is drinking may be combined by the processor with throat or other indicator sounds during or after the drinking indicator to detect choking or coughing.

In some embodiments, indicator breathing sounds with respect to the lungs, trachea, throat, nose, mouth, larynx may be used to detect whether a person is breathing, breathing rapidly, choking, coughing, sneezing, and the like. Such indicator sounds may also be used to determine whether a person has stopped breathing, is snoring, has sleep apnea, has sinus infection, allergies, hay fever, asthma, and the like. The frequency, amplitude, pattern, profile, timing, and location of such a sound may help determine any of these breathing issues, such as by identifying an indicator sound exceeds a threshold or includes a profile, such as noted herein. In some cases, a location of an allergic response by the wearer can be tracked to help people avoid risky allergy locations. For instance, using the motion sensor or another location sensor (e.g., of a cell phone) the location of the where can be identified and stored when the wearer experiences an allergic response. Such a response may be directly measured as a shift up in pitch (e.g., frequency) of breathing sounds detected by array 150, sensor 138, sensor 138B or other sensors described for sensing breathing sounds.

The sounds of snoring and or sleep apnea might easily be detected by the array (e.g., processor using output signals of sensors 138), and the array could facilitate determining the location of such a source of sound in the body of the wearer. Sounds of choking could be determined by the array even before an individual shows signs of distress. The distinct sound of a tooth cracking due to something that bitten into by the wearer might also be easily be detected by the array, and the array could facilitate determining the location of such a tooth in the body of the wearer. Next, the distinct sound of liquids that go down the wrong way (in trachea rather than esophagus) could also be easily detected by the array.

In some embodiments, selections of desired locations may be made by a user or wearer; or instructions from a designer, manufacturer or physician who sets up or otherwise programs the device (e.g., device 100, sensor 138, array 150, or a processor thereof) to perform the functions (e.g., detections of the indicator sound) noted above. The selection may consider or be based on the factors of performing the function noted with respect to that selection. In some cases, selections of desired locations may be based on known physiological, medical, or other knowledge and information.

In some cases, selections of indicator sound thresholds or profiles may be based on known physiological, medical, or other knowledge and information. Selections of indicator sound thresholds or profiles may be made by a user, wearer, designer, manufacturer or physician who sets up or otherwise programs the device (e.g., device 100, sensor 138, array 150, or a processor thereof) to perform the functions (e.g., detections of the indicator sound) noted above. The selection may consider or be based on the factors of performing the function noted with respect to that selection.

Some embodiments include a process for determining an indicator sound using array 510. Such a process may include locating sound sensors (e.g., sensors 138A-J or a subset of those sensors) at desired locations on a person based on the desire to sense an indicator sound. Locating the array of sound sensors may include locating sound sensors at desired locations to directly or indirectly (e.g., through triangulation) hear a sound expected to be heard from an organ of the person. Next, the sensors may sense a biometric environment and output electronic output signals from the sensors based on the sensed environment. In some cases, the sensors may sense sounds from organs of the person wearing the sensors. The output signals may be communicated by wired or wireless technology from the sensors and to a processor where they are received for processing. Next, the processor may output an alert signal to an alarm (e.g., speaker 114, vibrator 124, and/or a wireless signal such as to cellular phone 210) if an electronic output signal includes an indicator sound, is greater than a threshold or includes a profile sound. In some cases, an indicator sound includes an output signal indicating a sound is greater than the threshold or includes the profile sound. If an electronic output signal includes an indicator sound, the processor may determine (e.g., detect or indicate) damage to or other issues related to one of a heart, lungs, bone joint, jaw, mouth, nose, throat, veins or arteries of a person wearing the device based on the output signals. In response to receiving the alert signal, the alarm (e.g., speaker 114, vibrator 124, and/or cellular phone 210) may alerting the person wearing device 100 of the alert signal.

Embodiments may also include the ability to integrate (e.g., process by the processor) the signals from multiple sensors as well as different types of sensors to allow the integration of the sensor output signals which would allow for the detection of smaller shifts in indicators and reduce false positives. For example, smaller shifts or changes in the output signal of two sensors may be used to indicate an environment, biological event, or indicator sound due to combining the information of the small shifts of the two sensors at the same time. In one example, a smaller shifts or changes in the output signal of pressure sensor 134 and/or length sensor 132 (e.g., as compared to those for an alert due to these sensors as noted above) may be combined with an irregular profile heartbeat sound from sensor 138B to identify a heart attack.

Also, smaller shifts or changes in the output signal of two sensors may be used to reduce false positive indications of an environment, biological event, or indicator sound due to combining the information of the small shifts of the two sensors at the same time. In one example, the lack of a smaller shift or change in the output signal of pressure sensor 134 and/or length sensor 132 (e.g., as compared to those for an alert due to these sensors as noted above) may be combined with an irregular profile heartbeat sound from sensor 138B to determine that the irregular sound is not a heart attack (e.g., is due to issues with, poor location of, or damage to sensors 138B).

According to some embodiments, the microphone mesh (e.g., array 510) may be (e.g., instead of sound sensors 138) an array of small speakers at the desired locations. According to some other embodiments, the microphone mesh (e.g., array 510) may also include an array of small speakers (e.g., in addition to or as part of sensors 138). In these cases, the speakers may be programmed to be used as a sonic repellant. High frequency sounds are known to repel insects and mammals. Thus, the processor could be programmed to send to the speakers, audio signals at frequencies know to deter mosquitos (e.g., to reduce or eliminate a mosquito's interest, attraction or bites). The processor could also be programmed to send to the speakers, audio signals at frequencies know to deter dogs (e.g., reduce or eliminate a dog's interest, attraction or bites).

In some cases, the device herein is based on or fits into the form factor of a common belt or waistband (e.g., device 100 described below); or into a “mesh” or array of sound sensors (e.g., array 510 described below), and it is a full functional wearable device, so it can be easily used by waistband or clothing vendors or wearable device suppliers.

I some cases, device 100 is or includes a unique combination of components/techniques that provide an improvement over previously known structures and techniques by being (1) a fully integrated personal computer in a waistband, (2) which has a USB interface and can link with smart phones, pad computers and the like through applications (e.g., “Apps”), (3) monitoring respiration rate with higher accuracy than a non waistband design, (4) monitoring waist length, and helping the wearer control body shape and weight indirectly, (5) monitoring the abdomen pressure, which can help the wearer to control food quantity of each meal thus indirectly allowing the wearer to control weight, (6) tracking sitting time and generating vibration to remind the wearer to exercise for a while after a long sitting period (7) and/or tracking the frequency the wears goes to the bathroom and sending a vibration remind to the wearer to drink more water if the frequency is too small.

I some cases, array 510 is or includes a unique combination of components/techniques that provide an improvement over previously known structures and techniques by being (1) a fully integrated personal computer in a mesh or array, (2) which has a USB interface and can link with smart phones, pad computers and the like through applications (e.g., “Apps”), (3) monitoring whether there is or has been damage or other issues of the heart, lungs, bones, joints, jaw, throat, arteries, digestive tract, and the like.

### EXAMPLES

The following examples pertain to embodiments.

Example 1 is a wearable computer device capable of being worn around a person's waist, the device comprising: a computer processor disposed in a buckle; a flexible data bus electronically coupled to the processor and extending along a belt, the belt coupled to the buckle; a plurality of sensors disposed along the belt and electronically coupled to the data bus, the sensors configured to sense a biometric environment of a person wearing the device and output an output signal based on the sensed environment; the data bus to communicate the output signals from the sensors to the processor; an alarm coupled to the bus, the alarm capable of notifying a person of an alert; wherein the processor includes circuitry to receive the output signals and send an alert signal to the alarm if an output signal is greater than a threshold or includes a profile.

In Example 2, the subject matter of Example 1 can optionally include wherein the wearable computer device includes a waistband or belt; wherein the plurality of sensors are located at selected locations on an inside surface of the device to sense a physiological environment of the person; wherein the flexible data bus includes a flexible printed circuit board (PCB); wherein the data bus is to communicate electronic signals to the alarm from the processor; and wherein the alarm is to communicate the alert signal to the person.

In Example 3, the subject matter of Example 1 can optionally include wherein the sensors include a length sensor, a pressure sensor, and an accelerometer; and wherein the processor includes circuitry to receive the signals output by the sensors and determine respiration rate, waist length, food quantity of a meal, sitting or sleep time, and frequency of visits to the bathroom.

In Example 4, the subject matter of Example 1 can optionally include wherein the processor is configured or programmed to receive signals output by the pressure sensor, and one of: (1) determine a respiration rate of a person wearing the device based on the signals output by the pressure sensor, and send an indication of the rate to a device, or (2) determine whether an amount of food and drink consumed by a person wearing the device is greater than a threshold based on the signals, and send a too much food alert signal to the alarm if the amount is greater than a threshold.

In Example 5, the subject matter of Example 1 can optionally include wherein the processor is configured to receive signals output by the length sensor, and one of: (1) determine a waist length of a person wearing the device based on the signals output by the length sensor, and send a too large alert signal to the alarm if the waist length is greater than a threshold, or (2) determine a waist length of a person wearing the device based on the signals output by the length sensor over a period of time, and send a quick change alert signal to the alarm if the waist length changes by an amount greater than a threshold over the period of time.

In Example 6, the subject matter of Example 1 can optionally include wherein the processor is configured to receive signals output by the motion sensor, and one of: (1) determine motion of a person wearing the device over a period of time based on the signals output by the motion sensor, and send an exercise alert signal to the alarm if the motion is less than a threshold over the period of time, or (2) determine motion of a person wearing the device over a period of time at night based on the signals output by the motion sensor, and send a not enough good sleep alert signal to the alarm if the motion is greater than a threshold over the period of time.

In Example 7, the subject matter of Example 1 can optionally include wherein the processor is configured to receive signals output by the length and pressure sensors, and determine number of times a person wearing the device uses the bathroom over a period of time based on the signals output by the length and pressure sensors, and send a drink more water alert signal to the alarm if the number of times is less than a threshold over the period of time.

In Example 8, the subject matter of Example 1 can optionally include wherein the alert signal is transmitted by the processor to one of a vibrator disposed in the buckle, speaker disposed in the buckle, a universal serial bus (USB) port disposed in the buckle, or a wireless transceiver; and wherein the alert signal causes vibration of the vibrator, an alert sound by the speaker, a data transmission by the USB port, or a wireless alert signal transmission by the wireless transceiver to a smart phone.

In Example 9, the subject matter of Example 1 can optionally include a sound sensor disposed in the belt; wherein the processor is configured to receive signals output by the sound sensor, (1) to detect an indicator sound or profile sound of an organ of a person wearing the device over a period of time at night based on the signals output by the sound sensor, and send an alert signal to the alarm if the an indicator sound or profile sound is detected.

Example 10 is a method of determining a biometric reading comprising: locating a waistband including a processor around a waist of a person; sensing a biometric environment using the sensors; outputting electronic output signals from the sensors based on the sensed environment; communicating the output electronic signals from the sensors to a processor; outputting an alert signal to an alarm if an electronic output signal is greater than a threshold or includes a profile.

In Example 11, the subject matter of Example 10 can optionally include wherein locating the waistband includes locating the plurality of sensors at desired locations around the waist, and wherein outputting the alert signal includes alerting the person of the alert signal using an alarm.

In Example 12, the subject matter of Example 10 can optionally include wherein locating the waistband includes locating the plurality of sensors at desired locations around the waist, and wherein outputting the alert signal includes alerting the person of the alert signal using an alarm.

Example 13 is a method to a wearable computer device capable of being worn on person's body, the device comprising: a computer processor; a communications system to communicate with the processor; a plurality of sound sensors disposed in an array to be worn by the person, the sensors configured to sense sounds from a person wearing the device and output signals based on the sensed sounds; the communications system to communicate the output signals from the sensors to the processor; wherein the processor includes circuitry to receive the output signals and send an alert signal to the alarm if an output signal is greater than a threshold or includes a profile sound.

In Example 14, the subject matter of Example 13 can optionally include wherein the wearable computer device includes one of a mesh, clothing, or patches to be worn on the skin of the person; wherein the plurality of sensors are located at selected locations on an inside surface of the device to sense sounds from organs of the person; wherein the communications system is wired or wireless; further comprising an alarm to communicate the alert signal to the person.

In Example 15, the subject matter of Example 14 can optionally include wherein each sound sensor includes a carrier patch, a microphone, and one of a wired or wireless a communication module.

In Example 16, the subject matter of Example 13 can optionally include wherein the processor is configured or programmed to: (1) receive the output signals and determine one of damage to or other issues related to one of a heart, lungs, bone joint, jaw, mouth, nose, throat, veins or arteries of a person wearing the device based on the output signals, and send an indication of the rate to a device; and (2) determine whether an output signal of an organ of a person wearing the device is greater than a threshold or includes a profile sound over a period of time.

In Example 17, the subject matter of Example 13 can optionally include wherein the sound sensors are microphones having an audio input port directed towards the inside of the mesh and coupled to the processor through wires or through a wireless transceivers disposed in each microphone.

In Example 18, the subject matter of Example 13 can optionally include wherein the sensors have an antennae or other circuitry for determining their location relative to each other.

Example 19 is a method of determining an indicator sound comprising: locating an array of sound sensors at desired locations on a person based on sensing an indicator sound; sensing sounds from organs of the person using the sensors; outputting electronic output signals from the sensors based on the sensed sounds; communicating the output electronic signals from the sensors to a processor; outputting an alert signal to an alarm if an electronic output signal is greater than a threshold or includes a profile, or includes an indicator sound.

In Example 20, the subject matter of Example 19 can optionally include wherein locating the an array of sound sensors includes locating sensors at desired locations to directly or indirectly hear a sound expected to be heard from an organ of the person; wherein the indicator sound (1) includes an output signal indicating a sound is greater than a threshold or includes a profile sound, and (2) indicates damage to or other issues related to one of a heart, lungs, bone joint, jaw, mouth, nose, throat, veins or arteries of a person wearing the device; and further comprising using an alarm to alert the person of the alert signal.

In Example 21, the subject matter of Example 19 can optionally include wherein the sensed sounds include sounds from joints, footfalls and lungs, and wherein outputting an alert signal includes outputting an alert signal that the person is walking, jogging or running.

Example 22 is an apparatus comprising means for performing the method of any one of claims 10-12 and 19-21.

In the description above, for the purposes of explanation, numerous specific details have been set forth in order to provide a thorough understanding of the embodiments. It will be apparent however, to one skilled in the art, that one or more other embodiments may be practiced without some of these specific details. The particular embodiments described are not provided to limit embodiments of the invention but to illustrate it. The scope of the embodiments of the invention is not to be determined by the specific examples provided above but only by the claims below. In other instances, well-known structures, devices, and operations have been shown in block diagram form or without detail in order to avoid obscuring the understanding of the description. Where considered appropriate, reference numerals or terminal portions of reference numerals have been repeated among the figures to indicate corresponding or analogous elements, which may optionally have similar characteristics.

It should also be appreciated that reference throughout this specification to “one embodiment”, “an embodiment”, “one or more embodiments”, or “different embodiments”, for example, means that a particular feature may be included in the practice of the embodiments. Similarly, it should be appreciated that in the description various features are sometimes grouped together in a single embodiment, figure, or description thereof for the purpose of streamlining the disclosure and aiding in the understanding of various inventive aspects of embodiments. This method of disclosure, however, is not to be interpreted as reflecting an embodiment that requires more features than are expressly recited in each claim. Rather, as the following claims reflect, inventive aspects of embodiments that may lie in less than all features of a single disclosed embodiment. For example, although the descriptions and figures above describe forming a waistband and body mesh, the descriptions and figures above can be applied to forming other wearable devices or clothes such as a vest, shorts, socks, scarf, girdle, shirt and the like. Thus, the claims following the Detailed Description are hereby expressly incorporated into this Detailed Description, with each claim standing on its own as a separate embodiment of the invention.

1. A wearable computer device capable of being worn around a person's waist, the device comprising:
a computer processor disposed in a buckle;
a flexible data bus electronically coupled to the processor and extending along a belt, the belt coupled to the buckle;
a plurality of sensors disposed along the belt and electronically coupled to the data bus, the sensors configured to sense a biometric environment of a person wearing the device and output an output signal based on the sensed environment;
the data bus to communicate the output signals from the sensors to the processor;
an alarm coupled to the bus, the alarm capable of notifying a person of an alert;
wherein the processor includes circuitry to receive the output signals and send an alert signal to the alarm if an output signal is greater than a threshold or includes a profile.

2. The device of claim 1, wherein the wearable computer device includes a waistband or belt; wherein the plurality of sensors are located at selected locations on an inside surface of the device to sense a physiological environment of the person; wherein the flexible data bus includes a flexible printed circuit board (PCB); wherein the data bus is to communicate electronic signals to the alarm from the processor; and wherein the alarm is to communicate the alert signal to the person.

3. The device of claim 1, wherein the sensors include a length sensor, a pressure sensor, and an accelerometer; and wherein the processor includes circuitry to receive the signals output by the sensors and determine respiration rate, waist length, food quantity of a meal, sitting or sleep time, and frequency of visits to the bathroom.

4. The device of claim 1, wherein the processor is configured or programmed to receive signals output by the pressure sensor, and one of: (1) determine a respiration rate of a person wearing the device based on the signals output by the pressure sensor, and send an indication of the rate to a device, or (2) determine whether an amount of food and drink consumed by a person wearing the device is greater than a threshold based on the signals, and send a too much food alert signal to the alarm if the amount is greater than a threshold.

5. The device of claim 1, wherein the processor is configured to receive signals output by the length sensor, and one of: (1) determine a waist length of a person wearing the device based on the signals output by the length sensor, and send a too large alert signal to the alarm if the waist length is greater than a threshold, or (2) determine a waist length of a person wearing the device based on the signals output by the length sensor over a period of time, and send a quick change alert signal to the alarm if the waist length changes by an amount greater than a threshold over the period of time.

6. The device of claim 1, wherein the processor is configured to receive signals output by the motion sensor, and one of: (1) determine motion of a person wearing the device over a period of time based on the signals output by the motion sensor, and send an exercise alert signal to the alarm if the motion is less than a threshold over the period of time, or (2) determine motion of a person wearing the device over a period of time at night based on the signals output by the motion sensor, and send a not enough good sleep alert signal to the alarm if the motion is greater than a threshold over the period of time.

7. The device of claim 1, wherein the processor is configured to receive signals output by the length and pressure sensors, and determine number of times a person wearing the device uses the bathroom over a period of time based on the signals output by the length and pressure sensors, and send a drink more water alert signal to the alarm if the number of times is less than a threshold over the period of time.

8. The device of claim 1, wherein the alert signal is transmitted by the processor to one of a vibrator disposed in the buckle, speaker disposed in the buckle, a universal serial bus (USB) port disposed in the buckle, or a wireless transceiver; and wherein the alert signal causes vibration of the vibrator, an alert sound by the speaker, a data transmission by the USB port, or a wireless alert signal transmission by the wireless transceiver to a smart phone.

9. The device of claim 1 further comprising a sound sensor disposed in the belt; wherein the processor is configured to receive signals output by the sound sensor, (1) to detect an indicator sound or profile sound of an organ of a person wearing the device over a period of time at night based on the signals output by the sound sensor, and send an alert signal to the alarm if the an indicator sound or profile sound is detected.

10. A method of determining a biometric reading comprising:
locating a waistband including a processor around a waist of a person;
sensing a biometric environment using the sensors;
outputting electronic output signals from the sensors based on the sensed environment;
communicating the output electronic signals from the sensors to a processor; and
outputting an alert signal to an alarm if an electronic output signal is greater than a threshold or includes a profile.

11. The method of claim 10, wherein locating the waistband includes locating the plurality of sensors at desired locations around the waist, and wherein outputting the alert signal includes alerting the person of the alert signal using an alarm.

12. The method of claim 10, wherein the sensors include a length sensor, a pressure sensor, and an accelerometer; and further comprising:
receiving the output signals at the processor; and
the processor determining one of a respiration rate, waist length, food quantity of a meal, sitting or sleep time, or frequency of visits to the bathroom based on the output signals.

13. A wearable computer device capable of being worn on person's body, the device comprising:
a computer processor;
a communications system to communicate with the processor;
a plurality of sound sensors disposed in an array to be worn by the person, the sensors configured to sense sounds from a person wearing the device and output output signals based on the sensed sounds;
the communications system to communicate the output signals from the sensors to the processor;
wherein the processor includes circuitry to receive the output signals and send an alert signal to the alarm if an output signal is greater than a threshold or includes a profile sound.

14. The device of claim 13, wherein the wearable computer device includes one of a mesh, clothing, or patches to be worn on the skin of the person; wherein the plurality of sensors are located at selected locations on an inside surface of the device to sense sounds from organs of the person; wherein the communications system is wired or wireless; further comprising an alarm to communicate the alert signal to the person.

15. The device of claim 14, wherein each sound sensor includes a carrier patch, a microphone, and one of a wired or wireless a communication module.

16. The device of claim 13, wherein the processor is configured or programmed to: (1) receive the output signals and determine one of damage to or other issues related to one of a heart, lungs, bone joint, jaw, mouth, nose, throat, veins or arteries of a person wearing the device based on the output signals, and send an indication of the rate to a device; and (2) determine whether an output signal of an organ of a person wearing the device is greater than a threshold or includes a profile sound over a period of time.

17. The device of claim 13, wherein the sound sensors are microphones having an audio input port directed towards the inside of the mesh and coupled to the processor through wires or through a wireless transceivers disposed in each microphone.

18. The device of claim 13, wherein the sensors have an antennae or other circuitry for determining their location relative to each other.

19. A method of determining a biometric indicator sound of a person comprising:
locating an array of sound sensors at desired locations on the person based on sensing an indicator sound;
sensing sounds from organs of the person using the sensors;
outputting electronic output signals from the sensors based on the sensed sounds;
communicating the output electronic signals from the sensors to a processor; and
outputting an alert signal to an alarm if an electronic output signal is greater than a threshold or includes a profile, or includes an indicator sound.

20. The method of claim 19, wherein locating the an array of sound sensors includes locating sensors at desired locations to directly or indirectly hear a sound expected to be heard from an organ of the person;
wherein the indicator sound (1) includes an output signal indicating a sound is greater than a threshold or includes a profile sound, and (2) indicates damage to or other issues related to one of a heart, lungs, bone joint, jaw, mouth, nose, throat, veins or arteries of a person wearing the device; and
further comprising using an alarm to alert the person of the alert signal.

21. The method of claim 19, wherein the sensed sounds include sounds from joints, footfalls and lungs, and wherein outputting an alert signal includes outputting an alert signal that the person is walking, jogging or running.

22. (canceled)
